(19) United States
(12) Patent Application Publication
(10) Pub. No.: US 2019/0185854 A1
(43) Pub. Date: Jun. 20, 2019

(54) Micro-RNA Delivery Compositions, Devices, and Methods

(71) Applicants: Massachusetts Institute of Technology, Cambridge, MA (US); Ramot at Tel-Aviv University Ltd., Tel Aviv (IL)

(72) Inventors: Natalie Artzi, Brookline, MA (US); João Conde, Lisbon (PT); Noam Shomron, Herzelia (IL); Avital Gilam, Ra'anana (IL)

(21) Appl. No.: 16/312,620

(22) PCT Filed: Jun. 23, 2017
(86) PCT No.: PCT/US17/39072

§ 371 Date: Dec. 21, 2018

Related U.S. Application Data
(60) Provisional application No. 62/353,622, filed on Jun. 23, 2016.

(51) Int. Cl. C12N 15/113; A61K 47/69; A61K 33/243; A61K 9/06; A61P 35/04; A61K 47/62; A61K 47/59; A61K 47/61; A61M 5/19

(52) U.S. Cl. CPC C12N 15/113; A61K 47/6931; A61K 33/243; A61K 9/06; A61P 35/04; C12N 2310/3513; A61K 47/59; A61K 47/61; A61M 5/19; C12N 2310/141; C12N 2310/351; A61K 47/62

(57) ABSTRACT

Provided herein are compositions that include a metal nanoparticle functionalized with a miRNA and a targeting molecule. The compositions may be used to prevent or reduce the rate of metastasis of cancer cells. The compositions also may include a drug, such as a chemotherapeutic agent. The compositions also may include a hydrogel in which the metal nanoparticles are dispersed. Methods of miRNA and/or drug delivery and kits also are provided.

### CROSS-REFERENCE TO RELATED APPLICATIONS

This application claims priority to U.S. Provisional Patent Application No. 62/353,622, filed Jun. 23, 2016, which is incorporated herein by reference.

### BACKGROUND

Metastasis is a complex biological process, which requires cells to acquire motility abilities. Metastases are the primary cause for mortality in breast cancer, the most common cancer affecting women regardless of ethnicity (Weigelt, B. et al., Nat. Rev. Cancer 5, 591-602 (2005)). In fact, one in eight women is diagnosed with and develops invasive/metastatic breast cancer (Siegel, R. L. et al., CA Cancer J. Clin. 65, 5-29 (2015)).

Metastasis involves sequential steps that typically include (1) epithelial-to-mesenchymal transition, (2) local migration and invasion of cancer cells from the primary tumor to the surrounding host tissue, (3) intravasation into blood or lymphatic vessels, (4) dissemination via the blood or lymphatic stream, (5) extravasation to distant organ, (6) survival in dormancy, and, finally, (7) proliferation and angiogenesis within an organ. Only a unique subpopulation of primary tumor cells that acquires special traits (which allow the successful completion of all of these steps) can survive and produce secondary metastases. Therefore, each step in the metastasis process provides one or more potential targets for metastasis reduction or prevention.

Although metastasis is the primary cause for mortality in certain cancers, including breast cancer, current cancer therapies generally lack effective anti-metastatic strategies.

As regulators of gene expression, microRNAs (miRNAs) constitute an attractive candidate to control metastasis progression via regulating cell motility. miRNAs are non-coding small RNAs that negatively regulate gene expression, and are understood to be associated with tumorigenicity, invasion, and metastasis. Precise sequence complementation between the seed region, including bases 2-8 from the 5′ end of the miRNA, and its binding-site within the 3′ untranslated region (3′-UTR) of the target mRNA may be necessary to exert a downregulation effect. Recent studies have shown that germline sequence variants, such as single-nucleotide polymorphisms (SNPs) in miRNA-binding sites, can disrupt the downregulation by miRNAs, with a profound effect on gene expression levels and consequentially on the phenotype, which can lead to increased risk for cancer (see, e.g., Chin, L. J. et al., Cancer Res. 68, 8535-8540 (2008); Smits, K. M. et al., Clin. Cancer Res. 17, 7723-7731 (2011); and Zhang, L. et al., Proc. Natl Acad. Sci. USA 108, 13653-13658 (2011)). The effect of SNPs has prevented miRNAs from being used effectively to control metastasis.

Moreover, most nanomaterial research to date has focused on targeting a primary tumor, giving priority to systemic treatments, despite the promise and benefits of local and sustained therapies.

There remains a need for improved compositions and methods that are configured to prevent or reduce the rate of metastasis, treat a tumor in a local and/or sustained manner, or a combination thereof.

### BRIEF SUMMARY

Provided herein are improved compositions and methods for preventing or reducing the rate of metastasis, which rely, at least in part, on one or more functional roles of a miRNA as described herein. The compositions and methods herein also may be used to treat a tumor in a local and/or sustained manner.

In one aspect, compositions are provided that include a metal nanoparticle functionalized with a miRNA and a targeting biomolecule. The miRNA may be configured to bind to a gene at a target site including a germline sequence variant, and the targeting biomolecule may be configured to bind to a marker that is expressed or overexpressed by a cancer cell. The germline sequence variant, in one embodiment, includes a single nucleotide polymorphism. The gene to which the miRNA is configured to bind may include a PALLD gene, and the single nucleotide polymorphism may be rs1071738. The gene to which the miRNA is configured to bind may include (i) an ancestral allele that permits miRNA:mRNA binding, and (ii) an alternate allele that disrupts miRNA:mRNA binding; and the miRNA may include an engineered miRNA configured to bind to the alternate allele. The miRNA may include a wild-type miRNA, an engineered miRNA, or a combination thereof. The miRNA may include a wild-type miR-182, an engineered miR-182, a wild-type miR-96, an engineered miR-96, or a combination thereof. The compositions may also include a drug, such as one or more chemotherapeutic agents. The drug may be intercalated in the miRNA, functionalized to the metal nanoparticle, or a combination thereof. The compositions also may include a hydrogel in which the metal nanoparticle is dispersed. A drug also may be dispersed in the hydrogel, including a drug that is not associated with the functionalized metal nanoparticle.

In another aspect, methods of miRNA delivery are provided. In embodiments, the methods include providing a first solution including a first polymer component that includes a first polymer having one or more aldehydes; providing a second solution including at least one of (i) a dendrimer including at least two branches with one or more surface groups, wherein about 25% to 100% of the surface groups include at least one primary or secondary amine, and (ii) a second polymer component including a second polymer having one or more amines; combining the first and second solutions together to produce a hydrogel composite; and contacting one or more biological tissues with the hydrogel composite, wherein at least one of the first solution and the second solution includes a composition as described herein. At least one of the first solution and the second solution also may include a drug, such as one or more chemotherapeutic agents.

In a further aspect, kits for making a hydrogel composite are provided. In embodiments, the kids include a first part that includes a first solution including a first polymer component that includes a first polymer having one or more aldehydes; and a second part that includes a second solution including at least one of (i) a dendrimer that includes at least two branches with one or more surface groups, wherein about 25% to 100% of the surface groups include at least one primary or secondary amine, and (ii) a second polymer component including a second polymer having one or more amines, wherein at least one of the first solution and the second solution includes a composition as described herein. At least one of the first solution and the second solution also may include a drug, such as one or more chemotherapeutic agents. The kit may include a syringe, which may have separate reservoirs for the first solution and the second solution.

In an additional aspect, methods for local delivery of a miRNA to a biological tissue are provided. Embodiments of the methods include applying to a biological tissue a composition as described herein; and permitting a metal nanoparticle to diffuse from the composition into the biological tissue.

In yet another aspect, methods of treatment or prophylaxis of cancer in a patient are provided. In embodiments, the methods include administering to a patient in need thereof an effective amount of a composition as described herein; and binding the targeting biomolecule to a cancer cell to permit the miRNA to prevent or reduce the rate of metastasis of the cancer cell. The administering may include applying the composition locally to a tumor or to a tissue bed following resection of a tumor.

### BRIEF DESCRIPTION OF THE DRAWINGS

FIG. 1 depicts primary tumor volume following treatment with several embodiments of the compositions provided herein and free Cisplatin.

FIG. 2 depicts primary tumor weight following treatment with several embodiments of the compositions provided herein and free Cisplatin.

FIG. 3A depicts quantitative PCR (qPCR) determination of miR-96 expression levels in mice treated with embodiments of the compositions provided herein.

FIG. 3B depicts quantitative PCR (qPCR) determination of miR-182 expression levels in mice treated with embodiments of the compositions provided herein.

FIG. 3C depicts quantitative PCR (qPCR) determination of Palladin mRNA expression levels in mice treated with embodiments of the compositions provided herein.

FIG. 4 depicts a quantification of metastatic lung nodules at days 13, 20, and 27 after primary tumor induction in mice treated with embodiments of the compositions provided herein.

FIG. 5 depicts a quantification of mCherry emission at 620 nm from the lungs, liver, and brain of mice treated with embodiments of the compositions provided herein.

FIG. 6 depicts the mass range of spleens from mice treated with embodiments of the compositions provided herein and a control composition compared with non-treated mice (Sham).

FIG. 7 is a schematic representation of predicted binding sites for hsa-miR-182/96 on the 3′UTR of PALLD gene.

FIG. 8 depicts luciferase activities 48 hours following co-transfection with hsa-miR-182 or hsa-miR-96 in combination with embodiments of two PALLD 3′UTR constructs.

FIG. 9 depicts miR-182, miR-96, and Palladin expression levels in MCF-7 and Hs578 breast cancer cell lines.

FIG. 10 depicts the relative expression levels of Palladin isoform 4 (90 kDa).

FIG. 11 depicts the downregulation of endogenous Palladin mRNA expression following over-expression of hsa-miR-182 or hsa-miR-96 as assayed by qRT-PCR.

FIG. 12 depicts the decreased Palladin protein observed upon over-expression of hsa-miR-182 or hsa-miR-96.

FIG. 13 depicts Palladin mRNA up-regulation following downregulation of miR-182 or miR-96 by antago-miRs.

FIG. 14 depicts decreased Palladin protein levels upon stable over-expression of mmu-miR-182 or mmu-miR-96.

FIG. 15 depicts a rs1071738 SNP genotype of T47D human breast cancer cell line as determined by Sanger sequencing.

FIG. 16 depicts Palladin protein levels 48 hours following transfection of T47D cells by the indicated miRNAs, as determined by western blot analysis.

FIG. 17 depicts wound closure data collected from Hs578 cells transfected by either hsa-miR-182, hsa-miR-96 or pcDNA3 control plasmid (Ctrl).

FIG. 18 depicts the results achieved by over-expression of hsa-miR-182 and hsa-miR-96, as demonstrated by a Matrigel invasion assay.

FIG. 19 depicts the results of a transwell migration assay of 4T1 cells stably expressing mmu-miR-182 or mmu-miR-96.

FIG. 20 depicts the results of a Matrigel invasion assay of 4T1 cells expressing mmu-miR-182 or mmu-miR-96.

FIG. 21 depicts the effect on wound closure caused by the down-regulation of miR-96 and miR-182.

FIG. 22 depicts a quantification of lung area covered by a fluorescent signal in a tumor stably expressing miR-96 or miR-182.

FIG. 23 depicts the change in tumor volume in tumors stably expressing miR-96 or miR-182.

### DETAILED DESCRIPTION

In embodiments, the compositions provided herein are capable of treating a primary tumor, preventing or reducing the rate of metastasis, or a combination thereof. The compositions and methods may include delivering drug, miRNA, or a combination thereof. The miRNA may be capable of preventing metastasis or reducing the rate of metastasis. The miRNA may include a wild-type miRNA, an engineered miRNA, or a combination thereof.

In embodiments, metastasis is prevented or reduced, at least in part, by an engineered miR-96, an engineered miR-182, or a combination thereof (see Examples 7 and 8 herein), which is configured bind to a target site on the 3′-untranslated region (UTR) of a PALLD gene that includes the common functional variant rs1071738. This common functional variant is a single nucleotide polymorphism (SNP) within the miR-182 and miR-96 target sites that influences metastasis, including breast cancer metastasis, as described herein. Specifically, the PALLD SNP is a functional variant that abolishes miRNA:mRNA binding in an alternate allele, thereby leading to uncontrolled regulation of Palladin expression. Palladin expression may be controlled, however, by an engineered miRNA, such as an engineered miR-96 and/or an engineered miR-182, that is a complimentary miRNA that binds to the alternate allele. Therefore, the methods provided herein may include delivering at least one of a wild type miRNA and/or an engineered miRNA in vivo, such as an engineered miR-96 and/or an engineered miR-182, to prevent or reduce metastasis, thereby preventing or slowing cancers, such as breast cancer, from spreading to other organs and/or regions. The therapeutic anti-metastatic potential of Palladin modulation by administrating miRNA may be extended to many types of cancer, including pancreatic cancer. Moreover, exploiting the effects of a common germline sequence variant, as described herein, on gene expression and cancerous phenotype, may permit a more effective individualized anti-metastatic therapy.

Also provided herein are delivery compositions that may permit efficient, local, and/or sustained release of miRNA, as well as a combined therapy that relies on miRNA and one or more drugs, such as a chemotherapy drug. The combined therapy may improve clinical outcomes by promoting tumor shrinkage, preventing or slowing metastasis, or a combination thereof.

miRNAs

The compositions described herein generally include a metal nanoparticle conjugated with miRNA. One type of miRNA may be conjugated to a metal nanoparticle, or two or more types of miRNA may be conjugated to a metal nanoparticle. The miRNA conjugated to a metal nanoparticle may include one or more wild-type miRNAs, one or more engineered miRNAs, or a combination thereof. For example, a metal nanoparticle may be functionalized with at least one wild-type miRNA and at least one engineered miRNA, wherein the wild-type miRNA binds to an ancestral allele of a gene, and the engineered miRNA binds to an alternate allele of a gene.

A miRNA may include a thiol moiety. When a miRNA includes a thiol moiety, the miRNA may be conjugated to a metal nanoparticle via a sulfide bond. The thiol moiety may be located at a terminal position of a miRNA. When a metal nanoparticle is a gold nanoparticle, a thiolated miRNA may be bonded to the surface of the gold nanoparticle by the strong interaction of thiol groups (e.g., at the 5′ end of the miRNA oligos) with gold, forming a quasi-covalent interaction. Alternatively, a miRNA may be conjugated to a metal particle by other known techniques, e.g., through a linker, such as the drug linker and targeting biomolecule linker described herein.

The miRNA conjugated to a metal nanoparticle may be configured to bind to a gene at a target site including a germline sequence variant. The phrase “a target site including a germline sequence variant” includes a target site of a gene that is different among alleles. For example, the miRNA binding site of the ancestral allele of a PALLD gene is “a target site comprising a germline sequence variant”, because the alternate allele of a PALLD gene differs from the ancestral allele due to a single nucleotide polymorphism, and vice versa. The germline sequence variant, in embodiments, includes a single nucleotide polymorphism. In one embodiment, the gene encodes a cytoskeletal protein associated with cell-cell junctions, cell-matrix junctions, or a combination thereof. Examples of cytoskeletal proteins include, but are not limited to, Palladin, Vinculin, or a combination thereof. Examples of genes that encode a cytoskeletal protein include, but are not limited to, PALLD, ROCK2, KRT20, FGF7, ABR, MYLK, BCR, S100A8, CSF1R, EPHA3, PRKAR1A, PARVA, RHOG, CCDC88A, PDGFRB, TACC1, ACTG1, ADRA2A, BCL2, or a combination thereof. In a particular embodiment, the gene is selected from PALLD, ROCK2, S100A8, CSF1R, EPHA3, PARVA, PDGFRB, or a combination thereof.

In embodiments, the gene includes two or more alleles, and the miRNA includes an engineered miRNA configured to bind to at least one of the two or more alleles. For example, a gene may include an ancestral allele that permits miRNA:mRNA binding, and an alternate allele that disrupts miRNA:mRNA binding. Therefore, a wild-type miRNA may be used that binds to the ancestral allele, and an engineered miRNA that is complimentary to the alternate allele may be used to restore miRNA:mRNA binding. If the ratio of the ancestral allele and alternate allele differs among patients, then the ratio of the types of miRNA conjugated to the metal nanoparticle may be tailored. For example, if a patient's cells include a 20:80 ratio of ancestral allele to alternate allele, then a metal nanoparticle may be functionalized with a 20:80 mol ratio of a miRNA that binds to the ancestral allele and a miRNA that binds to the alternate allele.

In embodiments, the gene includes a PALLD gene, and the germline sequence variant is the single nucleotide polymorphism rs1071738.

In further embodiments, the gene includes a PALLD gene, the germline sequence variant is the single nucleotide polymorphism rs1071738, and the miRNA conjugated to the metal nanoparticle includes wild-type miR-182, an engineered miR-182, wild-type miR-96, an engineered miR-96, or a combination thereof. The engineered miR-182 and miR-96 may be prepared by replacing the G nucleotide corresponding to rs1071738 with a C nucleotide. This mutation may permit the seed regions of the resulting miRNAs to be fully complimentary to the binding site of the PALLD gene's alternate allele.

### Targeting Biomolecules

A metal nanoparticle may be functionalized with a targeting biomolecule that is configured to bind to a marker that is expressed or overexpressed by a diseased cell, such as a cancer cell. The marker may include a marker that is unique to the diseased cell. The targeting biomolecule, therefore, may result in the selective cellular uptake of the compositions provided herein or a component thereof in the target diseased cells. In one embodiment, a metal nanoparticle is functionalized with a miRNA; and, in another embodiment, a metal nanoparticle is functionalized with miRNA and a targeting biomolecule.

The marker to which a targeting biomolecule is configured to bind may include a fibrin-fibronectin complex. The diseased cell that expresses or overexpresses the marker may include a cancer cell, such as a 4T1 breast cancer cell.

The targeting biomolecules may generally include any targeting biomolecule, such as a peptide, that is configured to bind to one or more markers of diseased cells, such as markers that are unique to diseased cells, upregulated by diseased cells, or a combination thereof.

For example, the targeting biomolecule may include a peptide that targets cancer cells of a particular type. In one embodiment, the targeting biomolecule includes a pentapeptide. The pentapeptide may include CREKA (Cys-Arg-Glu-Lys-Ala). When the targeting biomolecule is or includes a peptide, the peptides may be a synthetic peptide. Not wishing to be bound by any particular theory, it is believed that synthetic peptides may result in higher stability when a metal nanoparticle is in a solution or liquid, and/or higher and more selective uptake.

A metal nanoparticle may be functionalized with one type of targeting biomolecule, or two or more types of targeting biomolecules. Each type of targeting biomolecule may be configured to bind to the same marker or different markers.

### Nanoparticles

In embodiments, the metal nanoparticle provided herein is functionalized with a miRNA and a targeting biomolecule. Generally, any ratio of miRNA to targeting biomolecule may be conjugated to the metal nanoparticle. In embodiments, the mol ratio of miRNA to targeting biomolecule conjugated to the metal nanoparticle is about 6:1 to about 1:0.5, about 4:1 to about 1:1, about 2:1 to about 1:1, or about 1.6-1.8:1.

The metal nanoparticle may be formed of any biocompatible metal or mixture of metals. The phrase “metal nanoparticle,” as used herein, refers to a particle having [1] an average diameter of about 1 nm to about 100 nm, and [2] a structure that includes at least 95%, by weight, of one or more metals. In one embodiment, the metal nanoparticle is a gold nanoparticle. The phrases “gold nanoparticle” or “gold nanoparticles” as used herein, refer to a particle or particles including gold in at least an amount of 50% by weight, and have an average diameter of about 1 nm to about 100 nm. In one embodiment, the gold nanoparticles include gold in an amount of at least 95% by weight. In another embodiment, the gold nanoparticles include gold in an amount of at least 99% by weight. In some embodiments, the uptake, in vivo biodistribution, or a combination thereof, may be controlled, at least in part, by selecting a particular size or sizes of a metal nanoparticle.

When the metal nanoparticle is a gold nanoparticle, the gold nanoparticle may be selected from those that are commercially available, or made by techniques known in the art, such as the citrate reduction method, e.g., see Lee, P. C. et al., J. Phys. Chem. 1982, 86(17), 3391-3395. When the citrate reduction method is used, the gold nanoparticles may include citrate groups on at least a portion of their surfaces. The citrate groups may be relied upon, at least in part, to functionalize the gold nanoparticles with a miRNA and a targeting biomolecule to form the metal nanoparticles provided herein. It is well-known, for example, that a thiol functional group can undergo an exchange with a citrate group on the surface of a gold nanoparticle.

In embodiments, a miRNA is present at an amount of about 200 mols to about 300 mols of miRNA per metal nanoparticle. In some embodiments, a miRNA is present at an amount of about 225 mols to about 275 mols of miRNA per metal nanoparticle. In further embodiments, a miRNA is present at an amount of about 250 mols of miRNA per metal nanoparticle.

In embodiments, a targeting peptide is present at an amount of about 50 mols to about 250 mols of targeting peptide per metal nanoparticle. In some embodiments, a targeting peptide is present at an amount of about 100 mols to about 200 mols of targeting peptide per metal nanoparticle. In further embodiments, a targeting peptide is present at an amount of about 145 mols to about 150 mols of targeting peptide per metal nanoparticle.

In embodiments, a miRNA is present at an amount of about 200 mols to about 300 mols of miRNA per metal nanoparticle, and a targeting peptide is present at an amount of about 50 mols to about 250 mols of targeting peptide per metal nanoparticle. In some embodiments, a miRNA is present at an amount of about 225 mols to about 275 mols of miRNA per metal nanoparticle, and a targeting peptide is present at an amount of about 50 mols to about 250 mols of targeting peptide per metal nanoparticle. In further embodiments, a miRNA is present at an amount of about 250 mols of miRNA per metal nanoparticle, and a targeting peptide is present at an amount of about 50 mols to about 250 mols of targeting peptide per metal nanoparticle.

In embodiments, a miRNA is present at an amount of about 200 mols to about 300 mols of miRNA per metal nanoparticle, and a targeting peptide is present at an amount of about 100 mols to about 200 mols of targeting peptide per metal nanoparticle. In some embodiments, a miRNA is present at an amount of about 225 mols to about 275 mols of miRNA per metal nanoparticle, and a targeting peptide is present at an amount of about 100 mols to about 200 mols of targeting peptide per metal nanoparticle. In further embodiments, a miRNA is present at an amount of about 250 mols of miRNA per metal nanoparticle, and a targeting peptide is present at an amount of about 100 mols to about 200 mols of targeting peptide per metal nanoparticle.

In embodiments, a miRNA is present at an amount of about 200 mols to about 300 mols of miRNA per metal nanoparticle, and a targeting peptide is present at an amount of about 145 mols to about 150 mols of targeting peptide per metal nanoparticle. In some embodiments, a miRNA is present at an amount of about 225 mols to about 275 mols of miRNA per metal nanoparticle, and a targeting peptide is present at an amount of about 145 mols to about 150 mols of targeting peptide per metal nanoparticle. In further embodiments, a miRNA is present at an amount of about 250 mols of miRNA per metal nanoparticle, and a targeting peptide is present at an amount of about 145 mols to about 150 mols of targeting peptide per metal nanoparticle.

The metal nanoparticle generally may have any shape or combination of two or more different shapes. For example, the metal nanoparticle may be in the shape of a sphere, a rod, or a combination thereof. In one embodiment, the metal nanoparticle is a nanosphere. The term “nanosphere,” as used herein, refers to nanoparticles having at least a substantially spherical shape, and an average diameter of about 1 nm to about 100 nm.

In embodiments, the average diameter of the metal nanoparticle is about 5 nm to about 100 nm, about 10 nm to about 100 nm, about 30 nm to about 50 nm, about 35 nm to about 45 nm, or about 40 nm. The average diameter of the nanoparticles herein may be determined by transmission electron microscopy (TEM) images. The use of the phrase “average diameter” should not be construed as implying that the metal nanoparticle is necessarily spherical in shape. When the metal nanoparticle is not at least substantially spherical in shape, the “average diameter” refers to the average largest dimension of the metal nanoparticle.

In one embodiment, the metal nanoparticle is a gold nanoparticle having an average diameter of about 5 nm to about 100 nm, about 10 nm to about 100 nm, about 30 nm to about 50 nm, about 35 nm to about 45 nm, or about 40 nm.

In one embodiment, the metal nanoparticle is a gold nanosphere having an average diameter of about 5 nm to about 100 nm, about 10 nm to about 100 nm, about 30 nm to about 50 nm, about 35 nm to about 45 nm, or about 40 nm.

The metal nanoparticle also may include a targeting biomolecule linker. A “targeting biomolecule linker” generally is any molecule that is covalently bonded to the metal nanoparticle and the targeting biomolecule. The targeting biomolecule linker, in embodiments, includes (i) a sulfur atom covalently bonded to the metal nanoparticle, and (ii) an ester moiety covalently bonded to the targeting biomolecule. The metal nanoparticle may include one or more types of targeting biomolecule linker. In one embodiment, the targeting biomolecule linker is a thiol-PEG-COOH targeting biomolecule linker, which has the following structure when the metal nanoparticle is conjugated to a targeting biomolecule:

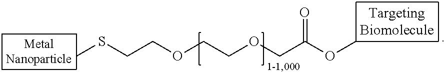

The thiol-PEG-COOH targeting biomolecule linker also may include one or more functional groups and/or moieties, including, but not limited to, an amide, a methylene group, an ether, an ester, or a combination thereof. For example, the thiol-PEG-COOH targeting biomolecule linker may include an amide and one or more methylene moieties. In one embodiment, the thiol-PEG-COOH targeting biomolecule linker includes an amide and five methylene moieties, and has the following structure: HS—C₂H₄—CONH-PEG-O—C₃H₆—COOH. The thiol-PEG-COOH targeting biomolecule linker may have a weight average molecule weight of about 2,000 g/mol to about 5,000 g/mol, about 3,000 g/mol to about 4,000 g/mol, or about 3,500 g/mol.

In embodiments, the targeting biomolecule includes a pentapeptide CREKA (Cys-Arg-Glu-Lys-Ala) with no modifications at the C- and N-terminals. The CREKA pentapeptide may be conjugated to a gold nanoparticle through a targeting biomolecule linker, such as a thiol-PEG-COOH targeting biomolecule linker.

The targeting biomolecule linker may be bonded to a targeting biomolecule, and then bonded to a metal nanoparticle, or a targeting biomolecule linker may be bonded to a metal nanoparticle, and then bonded to a targeting biomolecule. A metal nanoparticle generally may be functionalized with any amount of targeting biomolecule linker. In embodiments, a metal nanoparticle is functionalized with an amount of targeting biomolecule linker that allows for the addition of one or more thiolated molecules, such as thiolated-miRNAs.

### Compositions

Also provided herein are compositions that include a metal nanoparticle. The compositions provided herein may be dispersed in a medium. The medium may be any medium with which the metal nanoparticle is compatible, including media that aid in the handling and/or delivery of the metal nanoparticle. In one embodiment, the compositions also include a hydrogel in which the metal nanoparticle is dispersed. The hydrogel may include the contact product of the first solution and the second solution described herein. The metal nanoparticle and the may be substantially evenly or unevenly dispersed in the hydrogel.

### Drugs

The compositions provided herein may include a drug. The drug may include a single type of drug or two or more different types of drug. A drug may be (1) intercalated in a miRNA conjugated to a metal nanoparticle, (2) conjugated to a metal nanoparticle, (3) dispersed in a hydrogel, or (4) a combination thereof. A drug that is “dispersed in a hydrogel” may not be associated with a metal nanoparticle, either through functionalization or intercalation. In one embodiment, a drug is intercalated in a miRNA conjugated to a metal nanoparticle. In another embodiment, a drug is intercalated in a miRNA conjugated to a metal nanoparticle, and dispersed in a hydrogel. Not wishing to be bound by any particular theory, it is believed that the intercalation of a drug into miRNA conjugated to a metal nanoparticle may slow the release of drug from the compositions provided herein, and may result in a substantially continuous release of drug from the compositions provided herein. A drug may be intercalated in miRNA conjugated to a metal nanoparticle by contacting a metal nanoparticle functionalized with miRNA and a drug in a liquid. The liquid may include a first solution and/or second solution of the hydrogels described herein.

A drug is “conjugated to a metal nanoparticle” when it is covalently bonded to a metal nanoparticle, or to a drug linker that is covalently bonded to a metal nanoparticle.

A “drug linker” generally is any molecule that is covalently bonded to a metal nanoparticle and a drug. The drug linker, in embodiments, includes (i) a sulfur atom covalently bonded to a metal nanoparticle, and (ii) an ester moiety covalently bonded to a drug. A metal nanoparticle may include one or more types of drug linker. In one embodiment, the drug linker is a thiol-PEG-COOH drug linker, which has the following structure when a metal nanoparticle is conjugated to a drug:

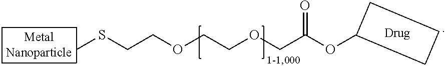

The thiol-PEG-COOH drug linker also may include one or more functional groups and/or moieties, including, but not limited to, an amide, a methylene group, an ether, an ester, or a combination thereof. For example, the thiol-PEG-COOH drug linker may include an amide and one or more methylene moieties. In one embodiment, the thiol-PEG-COOH drug linker includes an amide and five methylene moieties, and has the following structure: HS—C₂H₄—CONH-PEG-O—C₃H₆—COOH. The thiol-PEG-COOH drug linker may have a weight average molecule weight of about 2,000 g/mol to about 5,000 g/mol, about 3,000 g/mol to about 4,000 g/mol, or about 3,500 g/mol.

When a metal nanoparticle is functionalized with a drug, the drug that is conjugated to a metal nanoparticle may be active, i.e., exhibit a therapeutic effect, whether or not it remains covalently bonded to the metal nanoparticle and/or the drug linker. A drug may be configured to [1] remain conjugated to a metal nanoparticle, [2] be released from a metal nanoparticle to which it is conjugated, or [3] a combination thereof. The phrase “released from a metal nanoparticle to which it is conjugated,” as used herein, refers to the severing of one or more covalent bonds that conjugate the drug to the metal nanoparticle, either directly or through a drug linker. For example, a drug may be released from a metal nanoparticle to which it is conjugated upon the breaking of a covalent bond that [1] connects the metal nanoparticle to the drug linker, [2] connects the metal nanoparticle to the drug, [3] connects the drug linker to the drug, [4] exists between two or more atoms of the drug linker, or [5] a combination thereof. In one embodiment, a drug remains covalently bonded to the drug linker, or at least a portion thereof, upon the release of the drug from the metal nanoparticle to which it is conjugated. In another embodiment, a covalent bond connecting the drug and drug linker is severed upon release of the drug from the metal nanoparticle to which it is conjugated.

Generally, any drug may be included in the compositions described herein. Since the response to drugs can vary from patient to patient, the metal nanoparticle provided herein may be personalized on a patient-by-patient basis.

In embodiments, the drug may include one or more anti-angiogenic agents, one or more chemotherapeutic agents, or a combination thereof.

In embodiments, the drug includes one or more chemotherapeutic agents. A “chemotherapeutic agent” is a chemical compound useful in the treatment of cancer. In one embodiment, the drug includes one or more chemotherapeutic agents capable of intercalating into a miRNA conjugated to a metal nanoparticle.

In one embodiment, the chemotherapeutic agent includes cisplatin. Cisplatin is an alkylating agent classified as anti-neoplastic drug that has been extensively used in advanced breast cancer, especially in metastatic breast cancer and in triple-negative breast cancer. Moreover, the chemical structure of cisplatin permits it to intercalate into miRNA, which may result in a sustained release of cisplatin.

Examples of chemotherapeutic agents include, but are not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrirnidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. In embodiments, the drug includes an anti-angiogenic agent. An “anti-angiogenic agent” includes drugs that inhibit the growth of blood vessels. In one embodiment, the anti-angiogenic agent is bevacizumab (Avastie). Other anti-angiogenic agents that may be conjugated to the metal nanoparticles provided herein include, but are not limited to, axitinib, cabozantinib, cetuximab, everolimus, lenalidomide, pazopanib, ramucirumab, regorafenib, sorafenib, sunitinib, thalidomide, vandetanib, and zivaflibercept.

### Hydrogels

A metal particle described herein may be dispersed in a hydrogel. The metal nanoparticles described herein may be dispersed at least substantially evenly in a hydrogel, or unevenly in a hydrogel. The concentration of a metal nanoparticle in a hydrogel may be about 5 nM to about 50 nM, about 5 to about 30 nM, about 5 nM to about 20 nM, about 5 nM to about 15 nM, or about 10 nM.

The hydrogels described herein generally may include any biocompatible hydrogel. The hydrogel may serve as a matrix material for controlled delivery of miRNA and/or drug, localized miRNA and/or drug delivery, sustained delivery of miRNA and/or drug, or a combination thereof. Methods of locally delivering a miRNA and/or drug may include applying to a biological tissue, such as a human tissue, a miRNA and/or drug delivery composition as provided herein, and permitting at least one miRNA, drug, and/or metal nanoparticle to transport (e.g., diffuse) from the composition into the biological tissue. The hydrogel may adhere to one or more biological tissues, thereby reducing or eliminating the risk of unwanted material migration following application of the hydrogel to one or more selected tissue sites. The hydrogel generally may be degradable, injectable, or a combination thereof. A metal nanoparticle may be added to the hydrogel after hydrogel formation.

In embodiments, a miRNA along with a drug is delivered by applying to a tumor an adhesive hydrogel scaffold/patch in which metal nanoparticles functionalized with a miRNA are embedded.

The hydrogel may be used as a treatment or prophylaxis of cancer in a patient.

The hydrogel may include a contact product of [1] a first solution that includes the first polymer component described herein, and [2] a second solution that includes the second polymer component and/or the dendrimer component described herein. In embodiments, at least one of the first solution and the second solution includes a metal nanoparticle as described herein. Therefore, [1] the first solution may include a metal nanoparticle, [2] the second solution may include a metal nanoparticle, or [3] the first solution and the second solution may include a metal nanoparticle. The metal nanoparticle that is added to the first solution, second solution, or both the first solution and the second solution may be associated with a drug, as described herein. In other words, a drug may be (1) intercalated in miRNA conjugated to the metal nanoparticle, (2) conjugated to the metal nanoparticle, or (3) a combination thereof. When the metal nanoparticle is functionalized with a drug, an additional amount of the drug conjugated to the metal nanoparticle or one or more different types of drugs may be added to the first solution, the second solution, both the first solution and the second solution, or the hydrogel upon or after combining the first and second solutions. When a metal nanoparticle is not functionalized with a drug, a drug may be added to the first solution, the second solution, both the first solution and the second solution, or the hydrogel upon or after combining the first and second solutions. The drug may become associated with the metal nanoparticle (for example, via intercalation in miRNA conjugated to the metal nanoparticle) upon addition of the drug to a solution that also includes a metal nanoparticle, or upon or after the mixing of the first and second solutions when one of the solutions includes a metal nanoparticle and the other includes a drug.

A metal nanoparticle and/or drug may be added to the hydrogel after hydrogel formation. Therefore, a hydrogel may be formed, and a metal nanoparticle, drug, or combination thereof may be added to the hydrogel.

A metal nanoparticle may be present in the first solution, the second solution, or a combination thereof in an amount sufficient to impart the resulting hydrogel with a concentration of the metal particle of about 5 μM to about 75 μM, about 15 μM to about 65 μM, or about 25 μM to about 50 μM. The metal nanoparticle may be disposed in a solution having components with which the metal nanoparticle is incapable of reacting.

The rate of miRNA and/or drug delivery may be controlled, at least in part, by imparting the metal nanoparticle with one or more functional groups capable of reacting with a functional group of at least one component of the hydrogel in which the metal nanoparticle is dispersed. If the metal nanoparticle does not include functional groups capable of reacting with a functional group of at least one component of the hydrogel in which the metal nanoparticle is dispersed, then the rate of miRNA and/or drug delivery may be dictated by the diffusion of the metal nanoparticle a from the hydrogel. If the metal nanoparticle does include a functional group capable of reacting with a functional group of at least one component of the hydrogel, then the rate of miRNA and/or drug delivery may be dictated by the degradation rate of the hydrogel, the diffusion of the metal nanoparticle from the hydrogel, or a combination thereof.

Generally, the hydrogel composites and compositions, including miRNA and/or drug delivery compositions, provided herein may be formed by combining a first solution and a second solution as described herein. The first solution and the second solution may be aqueous macromer solutions. The first solution and/or the second solution may independently include water, phosphate buffer saline (PBS), Dulbecco's Modified Eagle's Medium (DMEM), or any combination thereof.

The first solution, in embodiments, includes a composition described herein and a first polymer component. The first solution, in other embodiments, includes a first polymer component without a composition described herein.

The second solution may include at least one of a dendrimer and a second polymer component. The dendrimer and/or second polymer component generally have one or more functional groups capable of reacting with the one or more functional groups on the first polymer. The second dendrimer and/or second polymer component, in particular embodiments, include one or more amines. The second solution, in other embodiments, also includes a composition as described herein or a component thereof.

The first solution and the second solution, in embodiments, are combined to form the hydrogel composites and compositions described herein. When combined, the aldehyde groups of the first solution may react with the amines that are present in the second solution. This reaction is referred to herein as “curing” or “gelling.”

In embodiments, the metal nanoparticle is substantially evenly (i.e., uniformly) dispersed in the first solution. In other embodiments, the metal nanoparticle is substantially evenly dispersed in the first solution and the second solution. In further embodiments, the metal nanoparticle is evenly dispersed in the second solution. Although the metal nanoparticle is evenly dispersed in preferred embodiments, other embodiments may not have an even dispersement of the metal nanoparticle.

In embodiments, the concentration of the metal nanoparticle in the first solution is about 0.01% to about 30% by weight of the first solution. In some embodiments, the concentration of the metal nanoparticle in the first solution is about 0.01% to about 25% by weight of the first solution. In further embodiments, the concentration of the metal nanoparticle in the first solution is about 0.01% to about 20% by weight of the first solution. In still further embodiments, the concentration of the metal nanoparticle in the first solution is about 0.01% to about 15% by weight of the first solution.

In embodiments, the concentration of metal nanoparticle in the second solution is about 0.01% to about 30% by weight of the second solution. In some embodiments, the concentration of the metal nanoparticle in the second solution is about 0.01% to about 25% by weight of the second solution. In further embodiments, the concentration of the metal nanoparticle in the second solution is about 0.01% to about 20% by weight of the second solution. In still further embodiments, the concentration of the metal nanoparticle in the second solution is about 0.01% to about 15% by weight of the second solution.

In embodiments, the concentration of the metal nanoparticle in the hydrogel composites or compositions described herein is about 0.01% to about 10% by weight of the hydrogel composite or composition. In some embodiments, the concentration of the metal nanoparticle in the hydrogel composites or compositions described herein is about 0.01% to about 8% by weight of the hydrogel composite or composition. In certain embodiments, the concentration of the metal nanoparticle in the hydrogel composites or compositions described herein is about 0.01% to about 6% by weight of the hydrogel composite or composition. In particular embodiments, the concentration of the metal nanoparticle in the hydrogel composites or compositions described herein is about 0.01% to about 5% by weight of the hydrogel composite or composition.

In embodiments, the concentration of first polymer component in the first solution is about 0.01% to about 40% by weight of the first solution. In further embodiments, the concentration of first polymer component in the first solution is about 0.01% to about 30% by weight of the first solution. In some embodiments, the concentration of first polymer component in the first solution is about 0.01% to about 20% by weight of the first solution. In a particular embodiment, the concentration of first polymer component in the first solution is about 20% by weight of the first solution. In additional embodiments, the concentration of first polymer component in the first solution is about 0.01% to about 10% by weight of the first solution. Typically, the concentration may be tailored and/or adjusted based on the particular application, tissue type, and/or the type and concentration of dendrimer and/or second polymer component used.

In embodiments, the concentration of the first polymer component in the hydrogel composites or compositions described herein is about 0.01% to about 20% by weight of the hydrogel composite or composition. In further embodiments, the concentration of the first polymer component in the hydrogel composites or compositions described herein is about 0.01% to about 15% by weight of the hydrogel composite or composition. In some embodiments, the concentration of the first polymer component in the hydrogel composites or compositions described herein is about 0.01% to about 10% by weight of the hydrogel composite or composition. In still further embodiments, the concentration of the first polymer component in the hydrogel composites or compositions described herein is about 0.01% to about 7% by weight of the hydrogel composite or composition.

In embodiments, the total concentration of dendrimer and second polymer component in the second solution is about 0.01% to about 40% by weight of the second solution. In further embodiments, the total concentration of dendrimer and second polymer component in the second solution is about 0.01% to about 30% by weight of the second solution. In some embodiments, the total concentration of dendrimer and second polymer component in the second solution is about 0.01% to about 20% by weight of the second solution. In additional embodiments, the total concentration of dendrimer and second polymer component in the second solution is about 0.01% to about 10% by weight of the second solution. In a particular embodiment, the total concentration of dendrimer and second polymer component in the second solution is about 25% by weight of the second solution. Typically, the concentration may be tailored and/or adjusted based on the particular application, tissue type, and/or the type and concentration of first polymer component used. As used herein, the phrase “total concentration of dendrimer and second polymer component” refers to the sum of the concentration of dendrimer and the concentration of the second polymer component. The phrase does not imply that both a dendrimer and a second polymer component must be present in the second solution. The second solution may include a dendrimer, second polymer component, or both a dendrimer and second polymer component.

In embodiments, the total concentration of dendrimer and second polymer component in the hydrogel composites or compositions described herein is about 0.01% to about 20% by weight of the hydrogel composite or composition. In further embodiments, the total concentration of dendrimer and second polymer component in the hydrogel composites or compositions described herein is about 0.01% to about 15% by weight of the hydrogel composite or composition. In some embodiments, the total concentration of dendrimer and second polymer component in the hydrogel composites or compositions described herein is about 0.01% to about 10% by weight of the hydrogel composite or composition. In still further embodiments, the total concentration of dendrimer and second polymer component in the hydrogel composites or compositions described herein is about 0.01% to about 7% by weight of the hydrogel composite or composition.

### First Polymer Component

The first polymer component generally includes a first polymer with one or more functional groups capable of reacting with one or more functional groups on a biological tissue and/or one or more functional groups on the dendrimer component and/or second polymer component of the second solution. The first polymer component, in embodiments, includes a first polymer having one or more aldehyde groups.

The polymers of the first polymer component may be selected from any biocompatible polymers capable of forming or imparting certain characteristics to the hydrogel composites and compositions described herein. The polymers of the first polymer component, for example, may be selected from at least one polysaccharide, at least one hydrophilic polymer, at least one hydrophobic polymer, or combinations thereof.

In one embodiment, the first polymer component includes a first polymer that is a polysaccharide having one or more aldehyde groups. In a certain embodiment, the first polymer component includes a first polymer that is a hydrophilic polymer having one or more aldehyde groups. In another embodiment, the first polymer component includes a first polymer that is a polysaccharide having one or more aldehyde groups, and a hydrophilic polymer. In further embodiments, the first polymer component includes a first polymer that is a polysaccharide having one or more aldehyde groups, a hydrophilic polymer, and a hydrophobic polymer. In some embodiments, the first polymer component includes a first polymer that includes a polysaccharide and a hydrophilic polymer, wherein both the polysaccharide and hydrophilic polymer have one or more aldehyde groups. Therefore, as used herein, the phrase “first polymer” refers to the one or more polymers of the first polymer component that include one or more functional groups, e.g., aldehydes, that are capable of reacting with a biological tissue and/or the functional groups of the dendrimer component and/or second polymer component. In still further embodiments, the first polymer component includes a first polymer that includes a polysaccharide and a hydrophilic polymer, wherein both the polysaccharide and hydrophilic polymer have one or more aldehyde groups, and a hydrophobic polymer.

In embodiments, the first polymer includes at least one polysaccharide. The at least one polysaccharide may be linear, branched, or have both linear and branched sections within its structure. The at least one polysaccharide may be anionic, cationic, nonionic, or a combination thereof. Generally, the at least one polysaccharide may be natural, synthetic, or modified—for example, by crosslinking, altering the polysaccharide's substituents, or both. In one embodiment, the at least one polysaccharide is plant-based. In another embodiment, the at least one polysaccharide is animal-based. In yet another embodiment, the at least one polysaccharide is a combination of plant-based and animal-based polysaccharides. Non-limiting examples of polysaccharides include, but are not limited to, dextran, dextrin, chitin, starch, agar, cellulose, hyaluronic acid, derivatives thereof, such as cellulose derivatives, or a combination thereof.

In embodiments, the at least one polysaccharide is nonionic. Non-limiting examples of nonionic polysaccharides include dextran, dextrin, and cellulose derivatives. In other embodiments, the at least one polysaccharide is anionic. Non-limiting examples of anionic polysaccharides include hyaluronic acid, chondroitin sulfate, alginate, and cellulose gum. In further embodiments, the at least one polysaccharide is cationic. The cationic character may be imparted by substituting the at least one polysaccharide with positively charge groups, such as trimethylammonium groups. Non-limiting examples of cationic polysaccharides include chitosan, cationic guar gum, cationic hydroxyethylcellulose, or other polysaccharides modified with trimethylammonium groups to confer positive charge.

In embodiments, the first polymer component includes one or more hydrophilic polymers. The hydrophilic polymers are modified, in some embodiments, to confer degradability. For example, the hydrophilic polymers may be modified with polyester groups in order to impart degradability of the hydrophilic polymer. In particular embodiments, the hydrophilic polymers are substituted with one or more functional groups, such as aldehydes, that are capable of reacting with biological tissue and/or the functional groups of the dendrimer and/or second polymer component, such as amines. Generally, any biocompatible hydrophilic polymer may be used. Non-limiting examples of hydrophilic polymers include poly(vinyl alcohol), poly(acrylic acid), poly(acrylamide), poly(ethylene oxide), or combinations thereof.

In embodiments, the first polymer component includes one or more hydrophobic polymers. The hydrophobic polymers may be modified with pendant hydrophilic polymers to adjust their characteristics. Non-limiting examples of hydrophobic polymers include polycaprolactam, poly(lactic acid), polycaprolactone, or combinations thereof.

In certain embodiments, the first polymer has a molecular weight of about 1,000 to about 1,000,000 Daltons. In one embodiment, the first polymer has a molecular weight of about 5,000 to about 15,000 Daltons. Unless specified otherwise, the “molecular weight” of the polymer refers to the number average molecular weight. The molecular weight may be adjusted to attain certain properties, as known to those of skill in the art.

Generally, the one or more functional groups of the first polymer may be present in a number sufficient to form the hydrogel composites and compositions described herein. In certain embodiments, the first polymer's degree of functionalization is adjustable. The “degree of functionalization” generally refers to the number or percentage of groups on the polymer that are replaced or converted to the desired one or more functional groups. The one or more functional groups, in particular embodiments, include aldehydes. In one embodiment, the degree of functionalization is adjusted based on the type of tissue to which the hydrogel composites or compositions is applied, the concentration(s) of the various components, and/or the type of polymer(s) or dendrimer(s) used in the first and second solutions. In one embodiment, the degree of functionalization is about 10% to about 75%. In another embodiment, the degree of functionalization is about 25% to about 60%. In yet another embodiment, the degree of functionalization is about 40% to about 50%.

In one embodiment, the first polymer is a polysaccharide having about 10% to about 75% of its vicinal hydroxyl groups converted to aldehydes. In another embodiment, the first polymer is a polysaccharide having about 25% to about 75% of its vicinal hydroxyl groups converted to aldehydes.

In one embodiment, the first polymer is dextran with a molecular weight of about 10 kDa. In another embodiment, the first polymer is dextran having about 50% of its vicinal hydroxyl group converted to aldehydes. In a further embodiment, the first polymer is dextran with a molecular weight of about 10 kDa and about 50% of its vicinal hydroxyl groups converted to aldehydes.

In some embodiments, a polysaccharide and/or hydrophilic polymer is oxidized to include a desired percentage of one or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization. In addition to, or independently of, other methods, aldehyde groups can be grafted onto the polymer backbone using known bioconjugation techniques in the event that oxidative methods are unsuitable.

### Second Polymer Component

The second polymer component generally includes a second polymer with one or more functional groups capable of reacting with one or more functional groups of the first polymer of the first polymer component. The second polymer component, in embodiments, includes a second polymer having one or more amines. The amines may be primary amines, secondary amines, or a combination thereof.

The polymers of the second polymer component may be selected from any biocompatible polymers capable of forming or imparting certain characteristics to the hydrogel composites and compositions described herein. The polymers of the second polymer component, for example, may be selected from at least one biopolymer, polyamine, or a combination thereof.

In one embodiment, the second polymer component includes a second polymer that is a biopolymer having one or more amines, such as primary amines, secondary amines, or a combination thereof. Non-limiting examples of biopolymers include chitosan, collagen, gelatin, other structural biomolecules, or a combination thereof. In a particular embodiment, the second polymer includes a polyamine. The polyamine may be synthetic. Non-limiting examples of polyamines include amine-terminated, multi-arm poly(ethylene oxide) and polyethyleneimine. In another embodiment, the second polymer component includes a second polymer that includes both (i) a biopolymer having one or more amines, and (ii) a polyamine. Therefore, as used herein, the phrase “second polymer” refers to the one or more polymers of the second polymer component that include one or more functional groups, e.g., amines, that are capable of reacting with the one or more functional groups of the first polymer component, such as aldehydes.

In some embodiments, the second polymer is a commercially available amine-terminated polymer, such as Type I collagen, Type II collagen, Type III collagen, gelatin that is acid- or base-catalyzed (i.e., Type A or Type B), or 10 kD dextran (Pharmacosmos A/S, Denmark).

### Dendrimer Component

In embodiments, the second solution includes a dendrimer component. The dendrimer component may include a dendrimer that may be substituted with one or more functional groups, such as amines, that are capable of reacting with the one or more functional groups of the first polymer of the first polymer component.

In some embodiments, the dendrimer has amines on at least a portion of its surface groups, which are commonly referred to as “terminal groups” or “end groups.” The dendrimer may have amines on from 25% to 100% of its surface groups. In some embodiments, the dendrimer has amines on 100% of its surface groups. In one embodiment, the dendrimer has amines on less than 75% of its surface groups. As used herein, the term “dendrimer” refers to any compound with a polyvalent core covalently bonded to two or more dendritic branches. In some embodiments, the polyvalent core is covalently bonded to three or more dendritic branches. In one embodiment, the amines are primary amines. In another embodiment, the amines are secondary amines. In yet another embodiment, one or more surface groups have at least one primary and at least one secondary amine.

In one embodiment, the dendrimer extends through at least 2 generations. In another embodiment, the dendrimer extends through at least 3 generations. In yet another embodiment, the dendrimer extends through at least 4 generations. In still another embodiment, the dendrimer extends through at least 5 generations. In a further embodiment, the dendrimer extends through at least 6 generations. In still a further embodiment, the dendrimer extends through at least 7 generations.

In one embodiment, the dendrimer has a molecular weight of about 1,000 to about 1,000,000 Daltons. In a further embodiment, the dendrimer has a molecular weight of about 3,000 to about 120,000 Daltons. In another embodiment, the dendrimer has a molecular weight of about 10,000 to about 100,000 Daltons. In yet another embodiment, the dendrimer has a molecular weight of about 20,000 to about 40,000 Daltons. Unless specified otherwise, the “molecular weight” of the dendrimer refers to the number average molecular weight.

Generally, the dendrimer may be made using any known methods. In one embodiment, the dendrimer is made by oxidizing a starting dendrimer having surface groups including at least one hydroxyl group so that at least a portion of the surface groups include at least one amine. In another embodiment, the dendrimer is made by oxidizing a starting generation 5 (G5) dendrimer having surface groups including at least one hydroxyl group so that at least a portion of the surface groups comprise at least one amine. In yet another embodiment, the dendrimer is made by oxidizing a starting G5 dendrimer having surface groups including at least one hydroxyl group so that about 25% to 100% of the surface groups include at least one amine. In a particular embodiment, the dendrimer is a G5 dendrimer having primary amines on about 25% to 100% of the dendrimer's surface groups. In a certain embodiment, the dendrimer is a G5 dendrimer having primary amines on about 25% of the dendrimer's surface groups.

In one embodiment, the dendrimer is a poly(amidoamine)-derived (PAMAM) dendrimer. In another embodiment, the dendrimer is a G5 PAMAM-derived dendrimer. In yet another embodiment, the dendrimer is a G5 PAMAM-derived dendrimer having primary amines on about 25% to 100% of the dendrimer's surface groups. In a further embodiment, the dendrimer is a G5 PAMAM-derived dendrimer having primary amines on about 25% of the dendrimer's surface groups.

In one embodiment, the dendrimer is a poly(propyleneimine)-derived dendrimer.

In some instances, at least one of the first solution, the first polymer component, the second solution, the second polymer component, and the dendrimer further includes one or more additives. Generally, the amount of additive may vary depending on the application, tissue type, concentration of the dendrimer in the second solution, the type of dendrimer, concentration of the second polymer component in the second solution, the type of second polymer component, the type of first polymer component, and/or the concentration of the first polymer component in the first solution. Example of suitable additives, include but are not limited to, pH modifiers, thickeners, antimicrobial agents, colorants, surfactants, and radio-opaque compounds. Specific examples of these types of additives are described herein. In one embodiment, at least one of the first solution, the first polymer component, the second solution, the second polymer component, and the dendrimer includes a foaming additive.

### Formation of Hydrogel Composites and Compositions

Generally, the hydrogel composites and compositions described herein may be formed by combining the first solution and the second solution in any manner. In some embodiments, the first solution, and the second solution are combined before contacting a biological tissue. In other embodiments, the first solution, and the second solution are combined, in any order, on or in a biological tissue. In further embodiments, the first solution is applied to a first biological tissue, the second solution is applied to a second biological tissue, and the first and second biological tissues are contacted. In still a further embodiment, the first solution is applied to a first region of a biological tissue, the second solution is applied to a second region of a biological tissue, and the first and second regions are contacted.

Generally, the hydrogel composites and compositions may be applied to a biological tissue as a miRNA and/or drug delivery composition. The hydrogel composites and compositions also may be configured as a tissue adhesive or sealant.

The hydrogel composites and compositions may be applied to the biological tissue using any suitable tool and methods. Non-limiting examples include the use of syringes or spatulas. Double barrel syringes with rigid or flexible discharge tips, and optional extension tubes, known in the art are envisioned. The hydrogel composites and compositions may be applied directly to a tumor or a tissue bed following tumor resection.

As used herein, the hydrogel composites and compositions are a “treatment” when they improve the response of at least one biological tissue to which they are applied. In some embodiments, the improved response is preventing or reducing the rate of metastasis, slowing or reversing tumor growth, eliminating or reducing the likelihood of cancer recurrence, inducing cytotoxicity in cancer cells, lessening overall inflammation, improving the specific response at the wound site/interface of the tissue and hydrogel composites or compositions, enhancing healing, repairing torn or broken tissue, or a combination thereof. As used herein, the phrase “lessening overall inflammation” refers to an improvement of histology scores that reflect the severity of inflammation. As used herein, the phrase “improving the specific response at the wound site/interface of the tissue and hydrogel composite or compositions” refers to an improvement of histology scores that reflect the severity of serosal neutrophils. As used herein, the phrase “enhancing healing” refers to an improvement of histology scores that reflect the severity of serosal fibrosis.

In embodiments, the hydrogel composites and compositions may be used in challenging or awkward implantation environments, including under flowing liquids and/or in inverted geometries.

Before or after contacting one or more biological tissues, the hydrogel composites and compositions may be allowed adequate time to cure or gel. When the hydrogel composites and compositions “cure” or “gel,” as those terms are used herein, it means that the one or more functional groups of the first polymer have undergone one or more reactions with the dendrimer and/or second polymer, and one or more biological tissues. Not wishing to be bound by any particular theory, it is believed that the hydrogel composites and compositions described herein are effective because the first polymer component reacts with both (i) the dendrimer and/or second polymer component, and (ii) the surface of the biological tissues. In certain embodiments, the first polymer component's aldehyde functional groups react with the amines on (i) the dendrimer and/or second polymer component, and (ii) the biological tissues to form imine bonds. In these embodiments, it is believed that the amines on the dendrimer and/or second polymer component react with a high percentage of the aldehydes of the first polymer component, thereby reducing toxicity and increasing biocompatibility of the hydrogel composites and compositions. Typically, the time needed to cure or gel the hydrogel composites and compositions will vary based on a number of factors, including, but not limited to, the characteristics of the first polymer component, second polymer component and/or dendrimer, the concentrations of the first solution and second solution, the pH of the first and second solution, and the characteristics of the one or more biological tissues. In embodiments, the hydrogel composites and compositions will cure sufficiently to provide desired bonding or sealing shortly after the components are combined. The gelation or cure time should provide that a mixture of the components can be delivered in fluid form to a target area before becoming too viscous or solidified and then once applied to the target area sets up rapidly thereafter. In one embodiment, the gelation or cure time is less than 120 seconds. In another embodiment, the gelation or cure time is between 3 and 60 seconds. In a particular embodiment, the gelation or cure time is between 5 and 30 seconds. The hydrogel may be at least partially cured into a shape, such as a disc, prior to being applied to one or more biological tissues.

In embodiments, the methods provided herein also include determining the amount and/or ratio of alleles of a gene to which an miRNA is configured to bind. This determination may be used to tailor the miRNA conjugated to a metal nanoparticle, as described herein.

### Tissue Specific Formulations

Generally, the hydrogel composites and compositions may be adjusted in any manner to compensate for differences between tissues. In one embodiment, the amount of first polymer component is increased or decreased while the amount of dendrimer and/or second polymer component is unchanged. In another embodiment, the amount of dendrimer and/or second polymer component is increased or decreased while the amount of first polymer component is unchanged. In another embodiment, the concentration of the first polymer component in the first solution is increased or decreased while the second solution is unchanged. In yet another embodiment, the concentration of the dendrimer and/or second polymer component in the second solution is increased or decreased while the first solution is unchanged. In a further embodiment, the concentrations of the both the first polymer component in the first solution and the dendrimer and/or second polymer component in the second solution are changed.

When the amine density on the surface of a particular biological tissue is unknown due to disease, injury, or otherwise, an excess of the first solution may, in some embodiments, be added when the hydrogel composites and compositions are first applied, then the amount of first solution may be reduced, e.g., incrementally or drastically, until the desired effect is achieved. The “desired effect,” in this embodiment, may be an appropriate or adequate curing time, adhesion, sealing, treatment, drug delivery, or a combination thereof. Not wishing to be bound by any particular theory, it is believed that an excess of the first solution may be required, in some instances, to obtain the desired effect when the amine density on a biological tissue is low. Therefore, adding an excess will help the user, in this embodiment, achieve adequate sealing or adhesion or treatment in less time.

In other embodiments, however, a lower amount of the first solution may be added when the hydrogel composites and compositions are first applied, then the amount of first solution may be increased, e.g., incrementally or drastically, until the desired effect is achieved, which may be adequate curing time, adhesion, sealing, treatment, or a combination thereof.

In embodiments, the hydrogel composites and compositions can be optimized in view of a target biological tissue, by adjusting one or more of the following: rheology, mechanics, chemistry/adhesion, degradation rate, drug delivery, and bioactivity. These can be adjusted, in embodiments, by altering the type and/or concentration of a metal nanoparticle, the type and/or concentration of the first polymer component, and type and/or concentration of the dendrimer, the type and/or concentration of the second polymer component, or a combination thereof.

### Hydrogel Composite and Composition Kits

In another aspect, a kit is provided that includes a first part that includes the first solution, and a second part that includes the second solution. The kit may further include an applicator or other device means, such as a multi-compartment syringe, for storing, combining, and delivering the two solutions and/or the resulting hydrogel composites and compositions to a tissue site.

In one embodiment, the kit includes separate reservoirs for the first solution and the second solution. In certain embodiments, the kit includes reservoirs for first solutions of different concentrations. In other embodiments, the kit includes reservoirs for second solutions of different concentrations.

In one embodiment, the kit includes instructions for selecting an appropriate concentration or amount of at least one of the first solution and/or second solution to compensate or account for at least one characteristic of one or more biological tissues. In one embodiment, the hydrogel composites and compositions are selected based on one or more predetermined tissue characteristics. For example, previous tests, may be performed to determine the number of density of bonding groups on a biological tissue in both healthy and diseased states. Alternatively, a rapid tissue test may be performed to assess the number or density of bonding groups. Quantification of tissue bonding groups can be performed by contacting a tissue with one or more materials that (1) have at least one functional group that specifically interacts with the bonding groups, and (2) can be assessed by way of fluorescence or detachment force required to separate the bonding groups and the material. In another embodiment, when the density of bonding groups on a biological tissue is unknown, an excess of the first polymer having one or more aldehydes, may be initially added as described herein to gauge the density of bonding groups on the surface of the biological tissue.

In certain embodiments, the kit includes at least one syringe. In one embodiment, the syringe includes separate reservoirs for the first solution and second solution. The syringe may also include a mixing tip that combines the two solutions as the plunger is depressed. The mixing tip may be release-ably securable to the syringe (to enable exchange of mixing tips), and the mixing tip may include a static mixer. In some embodiments, the reservoirs in the syringe may have different sizes or accommodate different volumes of solution. In other embodiments, the reservoirs in the syringe may be the same size or accommodate the same volumes of the solution.

In a further embodiment, one or more of the reservoirs of the syringe may be removable. In this embodiment, the removable reservoir may be replaced with a reservoir containing a first solution or second solution of a desired concentration.

In a preferred embodiment, the kit is sterile. For example, the components of the kit may be packaged together, for example in a tray, pouch, and/or box. The packaged kit may be sterilized using known techniques at suitable wavelengths (where applicable), such as electron beam irradiation, gamma irradiation, ethylene oxide sterilization, or other suitable techniques.

In the descriptions provided herein, the terms “includes,” “is,” “containing,” “having,” and “comprises” are used in an open-ended fashion, and thus should be interpreted to mean “including, but not limited to.” When methods and composite materials are claimed or described in terms of “comprising” various components or steps, the composite materials and methods can also “consist essentially of” or “consist of” the various components or steps, unless stated otherwise.

The terms “a,” “an,” and “the” are intended to include plural alternatives, e.g., at least one. For instance, the disclosure of “a metal nanoparticle,” “a pentapeptide,” “a targeting biomolecule”, and the like, is meant to encompass one, or mixtures or combinations of more than one metal nanoparticle, pentapeptide, targeting biomolecule, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in one embodiment, that the gold nanosphere has an average diameter of about 30 nm to about 50 nm. This range should be interpreted as encompassing average diameters in a range from about 30 nm to about 50 nm, and further encompasses “about” each of 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, and 49 nm, including any ranges and sub-ranges between any of these values.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

Unless otherwise noted, all statistical analyses were performed with Student's t-test. Results are represented as mean±s.e.m., unless noted otherwise. No animal or sample was excluded from the analysis. The P values are *P<0.05, **P<0.01 and ***P<0.005.

### Example 1—Synthesis of miRNA-Gold Nanoparticles

The nanoparticles (NPs) used in this examples included gold core having an average diameter of about 40 nm that was decorated with thiolated miRNAs and a targeting peptide.

Engineered miR-96 and miR-182 oligos were bound to the gold surface by the strong interaction of thiol groups (at the 5′ end of the miRNA oligos) and the gold core, forming a quasi-covalent interaction.

Thiolated-PEG-COOH enabled conjugation to 4T1-targeting peptide (CREKA) that was labeled with FITC. The pentapeptide CREKA (Cys-Arg-Glu-Lys-Ala) is a tumor-homing pentapeptide that specifically homes to fibrin-fibronectin complexes abundantly expressed in tumor microenvironment and that specifically binds to 4T1 breast cancer cells (see Zhou, Z., et al., Biomaterials 34, 7683-7693 (2013)).

Bare gold nanoparticles (AuNPs), with an average diameter of about 40 nm (about 7.15E+10 nanoparticles per mL) and an SPR peak at 530 nm (extinction coefficient 8.42E+09 M⁻¹ cm⁻¹, MW 3.91E+08 g mol⁻¹, surface area 5.03+03 nm²) were purchased from Cytodiagnostics.

The functionalization of PEGylated gold nanoparticles was carried out using commercial hetero-functional PEG functionalized with a 30% saturated surface of α-Mercapto-ω-carboxy PEG solution (HS—C₂H₄—CONH-PEG-O—C₃H₆—COOH, MW. 3500 Da, Sigma) (see, e.g., Sanz, V. et al., J. Nanopart. Res. 14, 1-9 (2012); and Conde, J. et al., ACS Nano 6, 8316-8324 (2012)). The 30% saturated PEG layer allowed the incorporation of additional thiolated components, such as a thiolated DNA-hairpin-Quasar 705 nm and a thiolated-oligo-BHQ2 quencher.

Briefly, 10 nM of the bare-gold nanoparticles were mixed with 0.006 mg ml⁻¹ of PEG solution in an aqueous solution of SDS (0.028%). After this, the mixture was incubated for 16 h at room temperature. Excess PEG was removed by centrifugation (15,000×r.p.m., 30 min, 4° C.).

The pentapeptide CREKA (Cys-Arg-Glu-Lys-Ala), with no modifications at the C- and N-terminals, was coupled to the PEG-AuNPs by a carbodiimide chemistry assisted by N-hydroxisuccinimide (EDC/NHS coupling reaction) between the carboxylated PEG terminal and the primary amine groups of the peptide. CREKA is a tumor-homing pentapeptide that specifically homes to fibrin-fibronectin complexes abundantly expressed in tumor microenvironments, and specifically binds to 4T1 breast cancer cells.

Briefly, 10 nM of nanoparticles-PEG, 1.98 mg ml⁻¹N-hydroxysulfosuccinimide (sulfo-NHS, Sigma) and 500 μg ml⁻¹ EDC (1-Ethyl-3-(3-dimethylaminopropyl)carbodiimide, Sigma) were incubated in 10 mM MES (2-(N-morpholino)ethanesulfonic acid, Sigma) at pH 6.2 and allowed to react for 30 min to activate the carboxylic groups. After this, activated nanoparticles were washed once with 10 mM MES, pH 6.2, and used immediately. The CREKA peptide was added to the mixture at a final concentration of 3 μg ml⁻¹ and allowed to react for 16 h at 25° C. The nanoparticles then were centrifuged at 20.000×g for 30 min at 4° C., and washed three times with Mili-Q water.

CREKA quantification was achieved using a Pierce BCA Protein Assay kit (Thermo Scientific) according to the manufacturer's instructions. Therefore, 0.025 mL of each standard and unknown sample (the supernatants) was mixed with 0.2 mL of the BCA Working Reagent (50:1, BCA reagent A:BCA reagent B) in each tube. The reaction mixture was incubated at 60° C. for 30 min. After this period, the tubes were cooled down to room temperature and the absorbance was measured at 562 nm. The standard curve was used to determine the CREKA concentration of each unknown sample (supernatant).

After peptide conjugation, thiolated miRNAs (miR-96, miR-182, and a scrambled miR from Dharmacon) were dissolved in 1 mL of 0.1 M DTT, extracted three times with ethyl acetate, and further purified through a desalting Illustra NAP-5 column Sephadex G-25 DNA grade (GE Healthcare), according to the manufacturer's instructions.

The purified thiolated miRNAs were incubated at a concentration of 10 μM, with an RNase-free solution of the peptide-PEG-AuNPs (10 nM) containing 0.08% SDS. Subsequently, the salt concentration was increased from 0.05 to 0.3 M NaCl with brief ultrasonication following each addition to increase the coverage of oligonucleotides on the nanoparticle surface. After the functionalization, which occurred for 16 h at 4° C., the particles were purified by centrifugation (20,000 g, 20 min, 4° C.), and re-suspended in diethyl pyrocarbonate (DEPC)-water. This procedure was repeated 3 times.

The number of miRNA strands per nanoparticle was quantified using a Quant-iT RiboGreen RNA Assay Kit, which is one of the most sensitive detection dyes for the quantitation of RNA in solution, with linear fluorescence detection in the range of 1-200 ng of RNA. The standard curve was used to determine the miRNA concentration of each unknown sample (supernatant). The following table summarizes the size and charge of all nanoparticles used in this Example, as well as the quantification of PEG, CREKA peptide, and miRNAs. All experiments were done in triplicate and the values are presented as mean±SEM.

| | | | | CREKA | |
|---|---|---|---|---|---|
| | Size | Zeta-Potential | PEG mol | peptide mol | miR mol |
| Nanoformulation | (nm) | (mV) | per particle | per particle | per particle |
| NPs-PEG | 43.7 ± 2.5 | −32.8 ± 2.4 | 804.6 ± 10.6 | NA | NA |
| NPs-PEG-Peptide | 45.8 ± 1.8 | −25.4 ± 4.5 | 804.6 ± 10.6 | 146.2 ± 3.8 | NA |
| NPs-PEG-Peptide | 51.0 ± 1.6 | −30.4 ± 2.1 | 804.6 ± 10.6 | 146.2 ± 3.8 | 248.3 ± 5.6 |
| miR-182 | | | | | |
| NPs-PEG-Peptide | 50.7 ± 2.1 | −30.2 ± 3.4 | 804.6 ± 10.6 | 146.2 ± 3.8 | 250.3 ± 4.4 |
| miR-96 | | | | | |
| NPs-PEG-Peptide | 50.9 ± 2.7 | −31.3 ± 2.9 | 804.6 ± 10.6 | 146.2 ± 3.8 | 251.8 ± 2.6 |
| Ctrl miR | | | | | |

With the remarkable loading capacity indicated by the foregoing table (miRNA:NP ratio around 250:1), the NPs of this example represent an efficient therapeutic route for miRNA delivery.

### Example 2—In Vitro miR-Nanoparticles Delivery

4T1 cells stably expressing mCherry were seeded at a density of 1×10⁵ cells per well in 24-well plates and grown for 24 h before incubation of nanoparticles (5 nM). On the day of incubation, the cells were about 50% confluent. For confocal microscopy, the cells were fixed with 4% paraformaldehyde in PBS for 15 min at 37° C., and stained with DAPI to allow nuclear staining and finally mounted in ProLong Gold Antifade Reagent (Invitrogen). Images of cells were taken with a Nikon AIR Ultra-Fast Spectral Scanning Confocal Microscope.

### Example 3—Nanoparticles-Hydrogel Scaffold Synthesis and Implantation

Tagged hydrogel scaffolds were developed by mixing equal parts of dendrimer amine of 12.5% solid content (Dendritech Inc.), and dextran aldehyde 5% solid content (Sigma-Aldrich) with 0.25% dextran (Sigma-Aldrich) to form 6 mm pre-cured disks.

For doped scaffolds, miRNA-nanoparticles (10 nM) of Example 1, and cisplatin (30 μM, Sigma-Aldrich) were added to the dendrimer solution before hydrogel formation.

All solutions were filtered through a 0.22 μm filter before hydrogel formation for in vivo implantation. Pre-cured disks of scaffold with nanoparticles were formed and implanted subcutaneously on top of the fat mammary tumor in BALB/c mice.

Non-invasive longitudinal monitoring of tumor progression was followed by scanning the mice with the IVIS Spectrum-bioluminescent and fluorescent imaging system (Xenogen XPM-2 Corporation) from mice bearing mammary tumors from 4T1 cells (n=5 animals per treated group).

Fifteen minutes before imaging, the mice were intraperitoneally injected with 150 μL of D-luciferin (30 mg ml⁻¹, Perkin Elmer) in DPBS (Lonza). Whole-animal imaging was performed at the indicated time points. Assessment of in vivo toxicity via mouse body weight evaluation was performed on all the animal groups during the 27 days after tumor induction, and the 20 days after hydrogel implantation.

Micro-CT images of the lungs were performed in an eXplore CT120-whole mouse MicroCT (GE Healthcare) at days 13, 20, and 27 after tumor induction. Histological sections of the tumors (n=5) were stained with haematoxylin and eosin, and for IHC analysis the tumors (n=5) were stained with anti-Ki67 antibody (Abcam ab15580, dilution 1:200), anti-Vinculin (Sigma cat#v4139, dilution 1:50) or anti-Palladin (Proteintech, cat#10853-1-AP, dilution 1:50) primary antibodies.

### Example 4—Development of Orthotopic Breast Cancer Mice Model

Due to the ability of the compositions of Examples 1 and 3 to deliver miRNA, the in vivo pharmacokinetic and therapeutic profiles of a miRNA NPs doped hydrogel scaffold were studied in an orthotopic metastatic breast cancer mouse model.

Tumors in the mammary fat pad were induced in BALB/c female mice by injection of 4T1 cells stably expressing mCherry. Hydrogel scaffolds loaded with the miRNA-NPs were implanted adjacent to the tumor in the mammary fat pad when tumors reached a desired volume (˜100 mm³, about 5 days after tumor induction). Seven days after hydrogel implantation the primary tumors were removed and the presence of metastases in the lungs was evaluated by micro-CT for additional 14 days. Then, mice were sacrificed and organs harvested and screened for the presence of macro-metastasis.

Specifically, tumors in the mammary fat pad were induced in BALB/c (AnNCrl, 6 weeks, Charles River) female mice by injection of 1×10⁶ 4 T1 cells stably expressing mCherry, suspended in 50 μL of HBBS (Lonza) solution. For determination of tumor growth, individual tumors were measured using caliper and tumor volume was calculated by the following equation: tumor volume (mm³)=width×(length²)/2. Treatments began when tumor volume reached about 100 mm³.

Primary tumor progression was measured by mCherry expression (emission at 620 nm) while the release of the miRNA-NPs was tracked fluorescently via live imaging system 7 days post-hydrogel implantation. Live imaging of mice and ex vivo fluorescent images of breast tumors revealed that FITC-labeled NPs were able to accumulate similarly in tumors from all treated groups.

This confirmed the capacity of this platform to provide an efficient in vivo miRNA mimic delivery. No signs of inflammation at the surgical site or changes in body weight were observed before or after breast tumor induction or hydrogel implantation, suggesting that the hydrogels and NPs are biocompatible with no or insignificant associated toxicity or side effects.

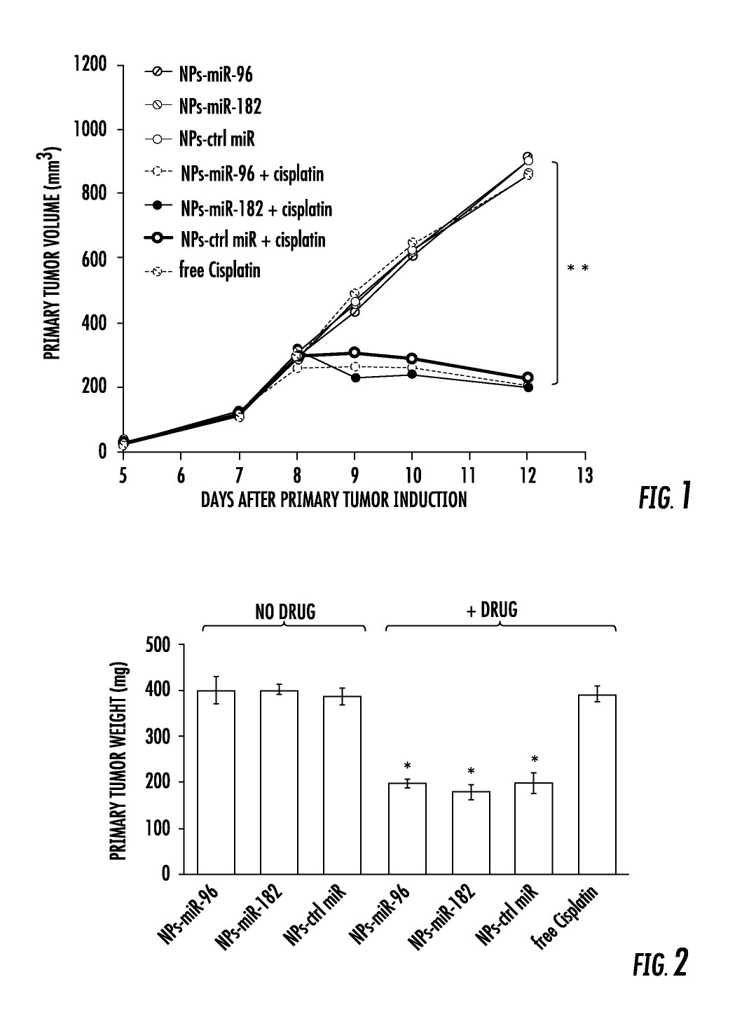

The hydrogel scaffolds doped with NPs carrying both miRNAs and the chemotherapeutic drug cisplatin showed lower mCherry expression in tumors compared to those carrying only miRNAs. This difference was demonstrated by mice live imaging, and ex vivo fluorescent images of breast tumors, which indicated efficient inhibition of the primary tumor's progression likely due at least in part to the release of cisplatin. A tumor size reduction of about 50% was observed for cisplatin-containing NPs 7 days after hydrogel disk implantation (FIG. 1), with a twofold reduction in tumor weight (FIG. 2).

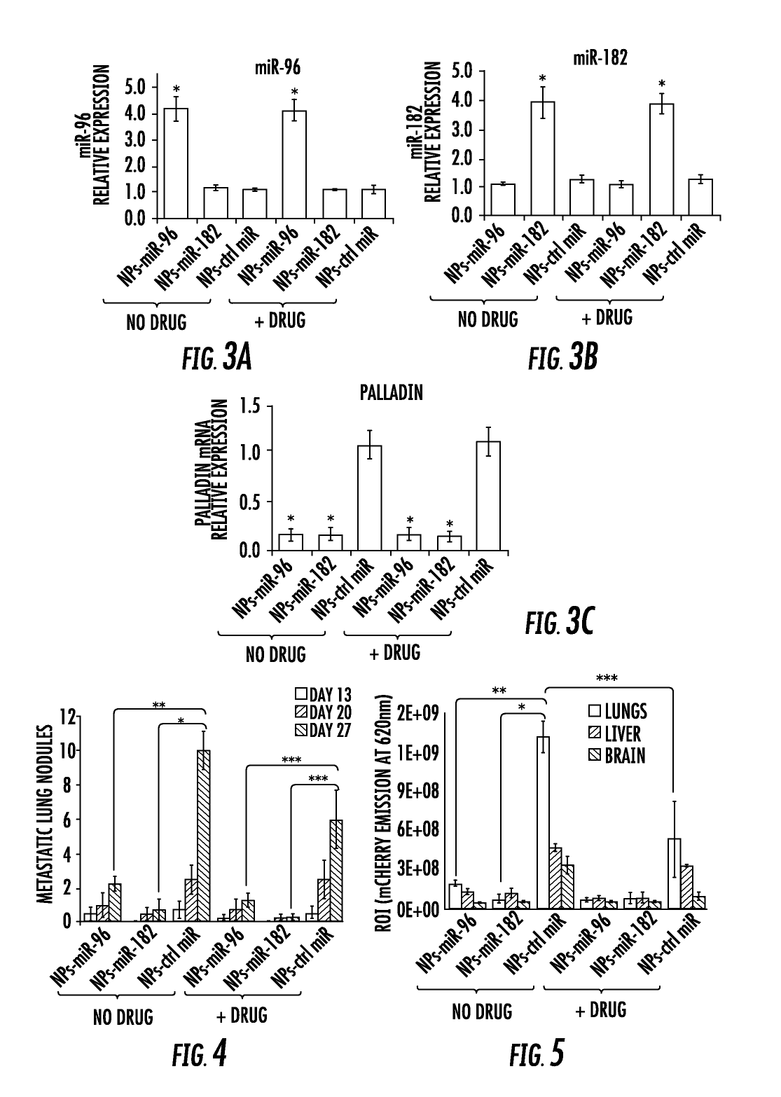

To evaluate the expression of miR-96 and miR-182, and their effect on Palladin expression, gene expression analysis of resected tumors was conducted. Real-time PCR results showed about a 4-fold increase in both miR-96 and miR-182 following treatment with hydrogels embedded with targeted NPs carrying miR-96 or miR-182 (with and without cisplatin), compared to the control miRNA (FIG. 3A, FIG. 3B, and FIG. 3C).

The tumors showed inverse expression levels of Palladin and the miRNAs. Palladin mRNA expression was (1) high only in groups treated with hydrogels embedded with targeted NPs carrying control miRNA, and (2) exhibited about a 5-fold decrease following treatment with miR-96 or miR-182 (FIG. 3C). Therefore, Palladin mRNA expression levels were suppressed by over-expression of miR-96 or miR-182.

Extensive reduction of vascularization in the cisplatin treated groups was evidenced by H&E staining of breast tumor sections, when compared with miRNA delivery only. Immuno-histochemical (IHC) analysis corroborated that the expression of Palladin and Vinculin (which also possess a conserved binding site for miR-96 and miR-182 on its 3′UTR, according to TargetScan) was extensively reduced when an over-expression of the miR-96 or miR-182 occurred, validating the qPCR results (FIG. 3A, FIG. 3B, and FIG. 3C).

In fact, both Palladin and Vinculin are cytoskeletal proteins that are associated with cell-cell and cell-matrix junctions, and required for organizing the actin cytoskeleton. Therefore, as the overexpression of miR-96 and miR-182 downregulated Palladin levels, a reduction in migration and invasion abilities occurred, as demonstrated by the in vitro assays described herein. Besides, IHC analysis of Ki-67, a cellular marker associated with cell proliferation, revealed that a decrease in this protein was mainly observed in groups treated with cisplatin, independent from the specific miRNAs treatment. This reveals that the treatment of the primary tumor with a chemotherapeutic drug reduces cancer cells proliferation, with a concomitant reduction in tumor size.

Knowing the potential invasive and metastatic profile of 4T1 cells, especially to lungs but also the liver and brain (Aslakson, C. J. et al., Cancer Res. 52, 1399-1405 (1992)), the effect was evaluated of enhancing miR-96 and miR-182 expression through local miRNA-mimic delivery on the establishment of in vivo metastasis. Metastasis formation was evaluated 13 days post-tumor resection using micro-CT of the lungs. The quantification of metastatic lung nodules in treated mice for days 13, 20, and 27 after primary tumor induction (which corresponded to 0, 7 and 14 days post tumor resection) revealed that the number of nodules increased with time only for groups treated with NPs carrying the control miRNA, and was more pronounced in the groups with no drug delivery (FIG. 4).

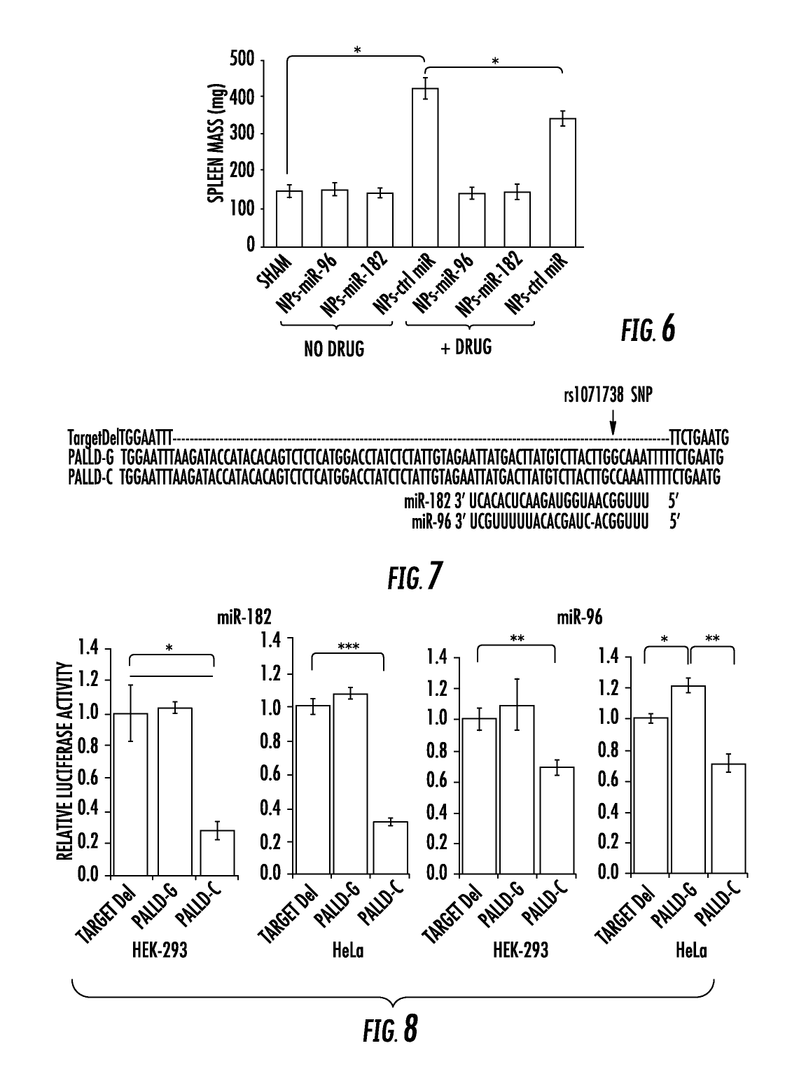

Ex vivo fluorescent images of lungs, liver, and brain depicting mCherry emission in treated mice revealed the presence of 4T1 cells (migrated from the mammary primary tumor), mainly in the groups treated with NPs carrying the control miRNA. H&E stains of the resected tumors revealed the presence of macro-metastasis in lungs only for mice treated with hydrogels embedded with targeted NPs carrying scrambled (Ctrl) miRNAs. In fact, the mCherry emission at 620 nm was higher in lungs, but also present in liver and brain mainly for mice treated with hydrogels embedded with targeted NPs carrying scrambled (Ctrl) miRNAs (FIG. 5). No metastases were detected in any other major organs.

In this example, only groups treated with NPs carrying the control miRNA, with or without the drug delivery, displayed enlarged (˜4-fold) spleens (i.e., splenomegaly) (FIG. 6). Mammary tumors induced with 4T1 cells are known for presenting splenomegaly, which is associated with a severe state of diseases, especially liver infections and some cancer types (DuPré, S. A., et al., Int. J. Exp. Pathol. 88, 351-360 (2007); and DuPré, S. A. et al., Exp. Mol. Pathol. 82, 12-24 (2007). The 4T1 tumor, according to testing that was conducted, appears to induce a leukemoid reaction with splenomegaly following orthotopic transplant into the mammary fat pads of female BALB/c mice.

### Example 5—Bioinformatics

To identify potential functional variants for breast cancer progression a stepwise omic-data integration approach was utilized. In step 1, a list of breast cancer genes (based on PubMed) was intersected with two additional datasets: TargetScan, a database of conserved miRNA target sites, and dbSNP, a database of known SNPs. Specifically, PubMed was searched with the term ‘breast cancer’ and gene name and gene symbol of all HUGO Gene Nomenclature Committee (HGNC)51 approved genes. Out of the 19,064 HGNC genes, a total of 7,608 had at least one publication with ‘breast cancer’ between years 2000 and 2013 (current year at the time). Genes with ≥4 publications (Q50=4) as breast cancer genes (n=4,057) were considered. Setting the cutoff to the median minimized weak associations with breast cancer (false positives), yet was sufficiently inclusive (4,057 of 19,064 HGNC genes, ˜20%). The following table depicts the results of step 1:

“Actin Cytoskeleton” Genes - Minor allele frequencies and publication on involvement in metastasis

| Gene. | | | | | | Supplementary | |
|---|---|---|---|---|---|---|---|
| Symbol | Chr | SNP.rsID | SNP.Position¹ | Allele | MAF² | reference #³ | Cancer Type |
| PALLD | Chr4 | rs1071738 | 169849389 | C, G, | 0.43 | | |
| ROCK2 | Chr2 | rs978906 | 11323276 | A, G, | 0.39 | 1-3 | osteosarcoma, |
| | | | | | | | hepatocellular |
| | | | | | | | carcinoma, |
| | | | | | | | cholangiocarcinoma |
| KRT20 | Chr17 | rs3169914 | 39032395 | A, G, | 0.37 | | |
| FGF7 | Chr15 | rs1057636 | 49776957 | A, C, | 0.37 | | |
| FGF7 | Chr15 | rs79465035 | 49777671 | —, A, | 0.23 | | |
| ABR | Chr17 | rs11247571 | 908502 | C, T, | 0.32 | | |
| MYLK | Chr3 | rs34709307 | 123331776 | —, A, | 0.27 | | |
| BCR | Chr22 | rs3876062 | 23658769 | A, G, | 0.24 | | |
| S100A8 | Chr1 | rs3006488 | 153362507 | A, G, | 0.17 | 4-6 | breast cancer, gastric |
| | | | | | | | adenocarcinoma, cervical |
| | | | | | | | cancer |
| CSF1R | Chr5 | rs3828609 | 149432863 | C, T, | 0.16 | 7 | breast cancer |
| EPHA3 | Chr3 | rs73139148 | 89530956 | A, G, | 0.13 | 8 | hepatocellular carcinoma |
| PRKAR1A | Chr17 | rs8905 | 66527802 | G, T, | 0.12 | | |
| PARVA | Chr11 | rs11022392 | 12551397 | A, G, | 0.11 | 9 | lung adempcarcinoma |
| RHOG | Chr11 | rs1049388 | 3848373 | C, G, | 0.08 | | |
| CCDC88A | Chr2 | rs17046829 | 55517219 | C.T. | 0.05 | | |
| PDGFRB | Chr5 | rs6674 | 149493535 | A, G, | 0.03 | 10 | mesenchymal-like |
| | | | | | | | colorectal |
| TACC1 | Chr8 | rs57661490 | 38707214 | C, T, | 0.01 | | |
| ACTG1 | Chr17 | rs1140892 | 79477356 | A, G, | 0.01 | | |
| ADRA2A | Chr10 | rs13306145 | 112839999 | A, G, | 0.01 | | |
| BCL2 | Chr18 | rs113207678 | 60794276 | A.T. | 0 | | |

¹SNP position based on February 2009 assembly of human genome (hg19)
²Minor Allele Frequency (MAF) in 1000 genomes (ALL) or in dbSNP when missing from 1000 genomes
³Reference for publication suggesting a role in metastasis for the specific gene

References: (1) Weigelt, B. et al., Nat. Rev. Cancer 5, 591-602 (2005); (2) Siegel, R. L. et al., CA Cancer J. Clin. 65, 5-29 (2015); (3) Gupta, G. P. et al., Cell 127, 679-695 (2006); (4) Chambers, A. F. et al., Nat. Rev. Cancer 2, 563-572 (2002); (5) Fidler, I. J. et al., Nat. Rev. Cancer 3, 453-458 (2003); (6) Weber, G. F., Cancer Lett. 328, 207-211 (2013); (7) Baranwal, S. et al., Int. J. Cancer 126, 1283-1290 (2010); (8) Chin, L. J. et al., Cancer Res. 68, 8535-8540 (2008); (9) Smits, K. M. et al., Clin. Cancer Res. 17, 7723-7731 (2011); and (10) Zhang, L. et al., Proc. Natl Acad. Sci. USA 108, 13653-13658 (2011).

In step II, the list of genes was further restricted to genes that were classified by the Gene-Ontology (GO) term ‘cytoskeleton organization’ since a critical step in tumor progression and metastasis is the acquisition of migration and invasion capabilities by reassembly of actin-cytoskeletal structures in the cell. Using this approach, 20 SNPs were identified that are located in 3′UTR miRNA-binding sites of 19 breast cancer genes known to be involved in cytoskeleton organization. Importantly, 6 of these genes (>30%) were previously identified as contributors to tumor metastases, as shown in the following table:

Association of Palladin and miR-96 and miR-182 expression with
lymph node metastases in The Cancer Genome Atlas (TCGA) Breast
invasive carcinoma cohort

| Lymph node metastases | Transcript | N | Effect (SEM)^{a} | P-value^{b} |
|---|---|---|---|---|
| Pathologic N | Palladin | 982 | 0.11 ± 0.04 | 5.20E−03 |
| (N0-3, ordinal) | miR-182 | 957 | 0.06 ± 0.04 | 1.22E−01 |
| | miR-96 | | −0.02 ± 0.04 | 5.83E−01 |
| Number of lymph nodes | Palladin | 854 | 0.06 + 0.02 | 2.61E−03 |
| (discrete) | miR-182 | 835 | 0.02 + 0.02 | 6.42E−01 |
| | miR-96 | | −0.01 ± 0.02 | 2.14E−01 |

^{a}The effect size represents the proportion of 1 SD change in standardized transcript residuals after adjustment for tumor stage (Pathologic N)
^{b}P-values were calculated using ANOVA

Specifically, the breast cancer genes were restricted to genes with conserved miRNA target sites in their 3′-UTR based on TargetScan (11,161 genes with conserved miRNA target in db) 52, resulting in 2,602 genes, and in step 3, the genes were restricted to those with a common (≥1%) SNP located in the miRNA target sites based on the dbSNP138 common database (12,896, 132 SNPs in db), resulting in a total of 190 genes and 212 variants.

The R package ‘RISmed’ was used to retrieve information from PubMed. The RefSeq, and dbSNP138 common databases were downloaded from the UCSC genome annotation database for the February 2009 assembly of the human genome (hg19), and overlaps between genomic intervals were calculated by the R package ‘GenomicFeatures’. The final gene list was annotated by Gene Ontology biological process classifications using the R packages ‘clusterProfiler’. Variants from genes classified by the term ‘cytoskeleton organization’ (n=19) were considered as potential functional variants for breast cancer progression to metastasis.

Of the potentially functional SNPs found in ‘cytoskeleton organization’ genes, focus was placed on the SNP with the highest (>43%) Minor Allele Frequency (MAF), and thus the largest population effect, rs1071738 in the PALLD gene. It was hypothesized that this type of functional variant is not under strong negative selection as its effect is exerted after the reproduction period.

The PALLD gene encodes Palladin cytoskeletal associated protein, which was recently shown to be involved in the invasive behavior of metastatic cells, specifically breast cancer cells, by increasing migration and invasive motility, through regulation of podosome and invadopodia protrusions formation (see, e.g., Lambrechts, A., et al., Int. J. Biochem. Cell Biol. 36, 1890-1909 (2004); and Cannon, A. R. et al., Cytoskelet. Hoboken N.J. 72, 402-411 (2015)).

The ancestral C allele of rs1071738 is the minor allele in dbSNP and in European populations, and the alternate G allele is the major allele. However, the allelic frequency can vary between diverse populations (20-90%).

The PALLD SNP was located within a predicted binding site for miR-96 and miR-182. The ‘seed’ regions of miR-96 and miR-182 are fully complimentary when the ancestral C allele is present at their binding sites, and harbor one mismatch when the alternate G allele is present (FIG. 7).

### Example 6—Cell Culture

HEK-293 T, HeLa, Hs578, MCF-7 and T47D cell lines were cultured in DMEM supplemented with 10% FBS (GIBCO) and 1% L-glutamine, 100 units per mL penicillin, and 100 units ml⁻¹ streptomycin (Biological Industries, Kibbutz Beit Haemek, Israel). The 4T1 cell line was cultured in RPMI (GIBCO) supplemented with 10% FBS (GIBCO), 1% L-glutamine, 1 mM sodium pyruvate, 100 units per mL penicillin, 100 units per mL streptomycin, 10 mM HEPES buffer (Biological Industries, Kibbutz Beit Haemek, Israel) and 2.5 g l⁻¹ D-Glucose (Sigma).

Cells were incubated at 37° C. in 5% CO₂ atmosphere. Hs578, MCF-7, and T47D cell lines were received from Prof. Ilan Tsarfaty (Tel-Aviv University). The 4T1 cell-line was received from Prof. Ronit Satchi-Fainaro (Tel-Aviv University). HeLa and HEK-293 T cell-lines were purchased from the American Type Culture Collection (ATCC). STR profiling (DNA Diagnostics Centre, UK) and mycoplasma testing (Biological Industries) were conducted for each cell line before use.

### Example 7—Regulation of Palladin Expression and Engineered miRNAs

To validate the regulation of Palladin expression by miR-96 and miR-182, a luciferase reporter assay was performed. Significant direct down-regulation of Palladin by miR-96 (about 30% reduction) and miR-182 (about 70% reduction) was observed in the presence of the complementary C allele (in both HeLa and HEK-293T cell lines) (FIG. 8).

However, in the presence of the alternate G allele, Palladin regulation by miR-96/182 was substantially abolished.

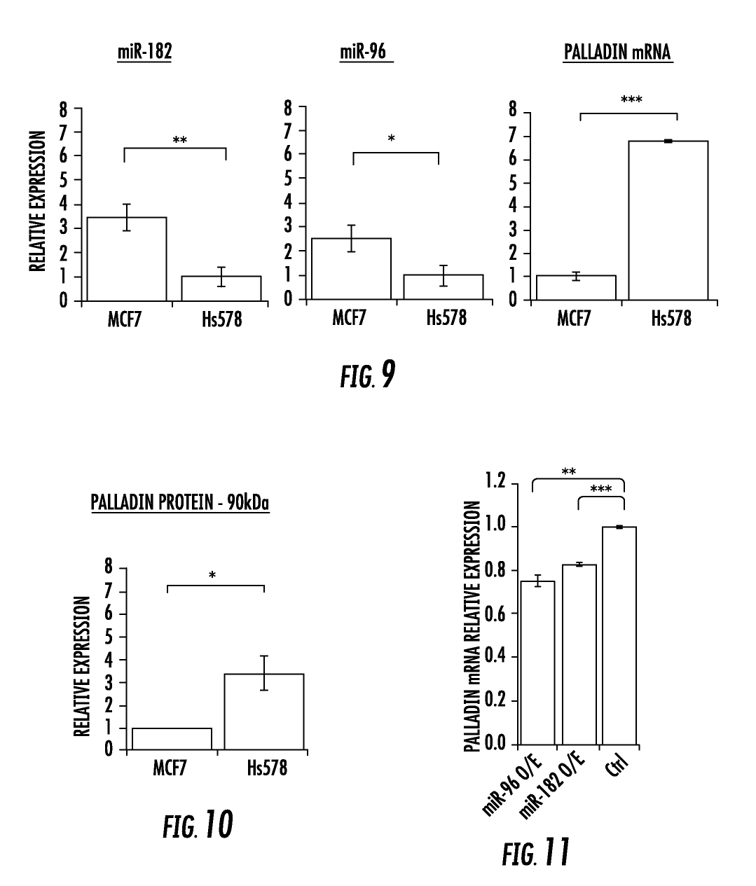

Palladin and miRs' expression in vitro in two human breast cancer cell lines was determined. The two human breast cancer cell lines were (1) MCF-7, a non-invasive breast cancer cell line, and (2) Hs578, a highly invasive breast cancer cell line. Both of these cell-lines are heterozygotes for rs1071738. These cell-lines showed opposite expression profiles of Palladin and the miRNAs. In the invasive cell-line, Hs578, the expression of Palladin was relatively high, and miR-96 and miR-182 was low. In the non-invasive MCF-7 cell-line, the opposite trend was observed (FIG. 9 and FIG. 10), further supporting down-regulation of Palladin expression by miR-96/182. FIG. 9 depicts miR-182, miR-96, and Palladin expression levels in MCF-7 and Hs578 breast cancer cells lines. RNA was extracted from MCF-7 and Hs578 cells lines, and the expression levels of hsa-miR-182, hsa-miR-96, and Palladin mRNA were assayed by qRT-PCR. miRNA levels were normalized to U6, and mRNA expression levels were normalized to GAPDH levels. FIG. 10 depicts the relative expression levels of Palladin isoform 4 (90 kDa).

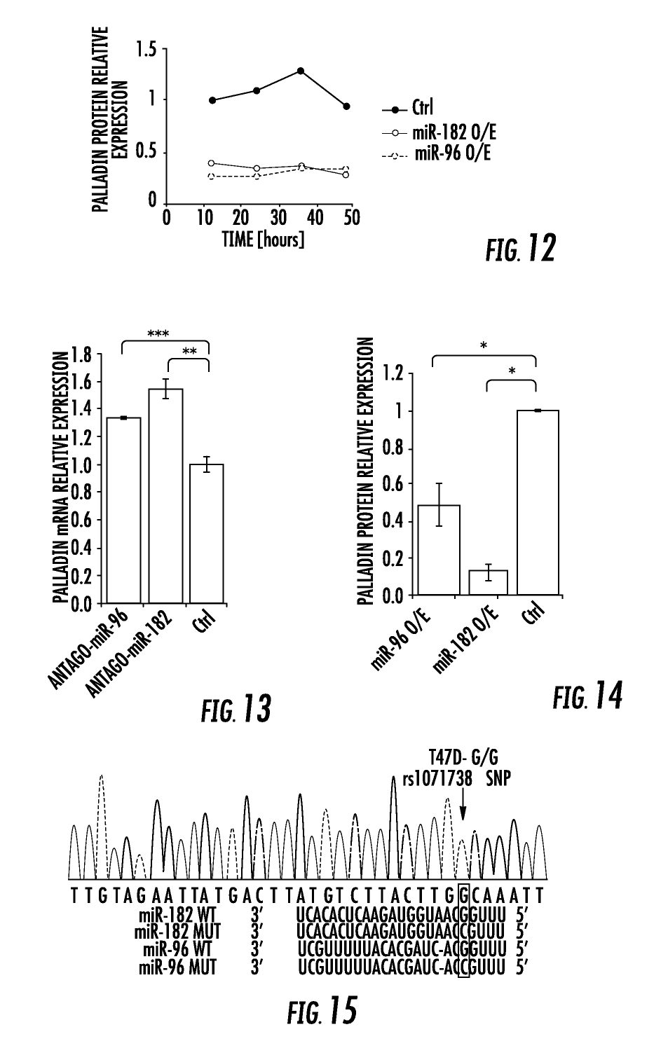

Palladin expression levels were suppressed by over-expression of miR-96 or miR-182 in Hs578 cells (mRNA and protein in FIG. 11 and FIG. 12, respectively), and increased following miR-96/182 down-regulation in MCF-7 cells (FIG. 13). FIG. 11 depicts the downregulation of endogenous Palladin mRNA expression following over-expression of hsa-miR-182 or hsa-miR-96 as assayed by qRT-PCR. RNA was extracted from Hs578 cells 24 hours following over-expression of either hsa-miR-182, hsa-miR-96 or pcDNA3 control plasmid. mRNA expression levels were normalized to GAPDH. The data at FIG. 12 was collected by extracting protein from Hs578 cells at the indicated time points following transfection. pcDNA3 plasmid was used as control. Bands quantification was done using ImageJ software and protein levels were normalized to Actin levels. FIG. 13 depicts Palladin mRNA up-regulation following downregulation of miR-182 or miR-96 by antago-miRs, as assayed by qRT-PCR. RNA was extracted from MCF7 cells 24 hours following transfection with either antago-miR-182, antago-miR-96 or scrambled control. mRNA expression levels were normalized to GAPDH.

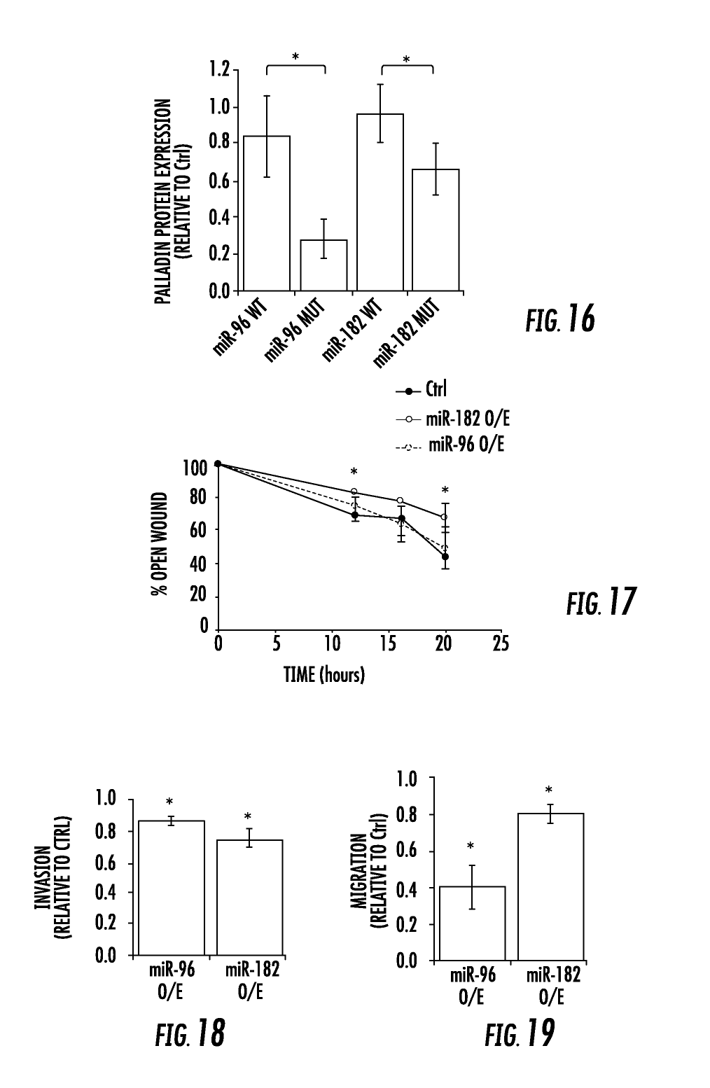

To further examine the mechanism of this effect in vitro, the highly aggressive mice breast cancer cell-line, 4T1, was utilized, which is a homozygote for the ancestral C allele of rs1071738. The binding-site of miR-96 and miR-182 at the region complementary to the ‘seed’ is identical and evolutionarily conserved between the human and mouse Palladin orthologous. The miR-96 sequence is identical to humans, and the miR-182 sequence differs in two nucleotides at the 3′ end of the miRNA. In agreement with the human results, inducing stable expression of mouse miR-96 or miR-182 reduced Palladin levels dramatically in 4T1 cells (FIG. 14). The prominent reduction in Palladin levels following over-expression of miR-96 or miR-182 was likely caused by stronger binding to the C alleles at the binding site, and/or by stable expression of the miRNAs. FIG. 14 depicts decreased Palladin protein levels upon stable over-expression of mmu-miR-182 or mmu-miR-96. Protein was extracted from 4T1 cells 2 to 3 weeks following infection. Scrambled plasmid was used as control. Bands quantification was performed using ImageJ software and protein levels were normalized to Actin levels.

The genotype dependent dysregulation was then ‘repaired’ by applying a complimentary engineered miRNA. Using the T47D human breast cancer cell line, which is a homozygote for the alternate G allele, it was observed that over-expression of WT miR-96 or miR-182 did not influence Palladin levels, whereas over-expression of engineered miR-96 or miR-182 in which the G nucleotide on the opposed position of the SNP was replaced by a C nucleotide, thereby allowing full complementation with the binding site (FIG. 15), repaired binding and reduced Palladin levels (FIG. 16). These results demonstrate a functional regulatory effect for the rs1071738 SNP, in which the ancestral C allele permits miRNA:mRNA binding and the alternate G allele disrupts binding. FIG. 15 depicts a rs1071738 SNP genotype of T47D human breast cancer cell line as determined by Sanger sequencing. Aligned are 4 sequences: two mature wild-type (WT) hsa-miR-182 and hsa-miR-96 that possess a G nucleotide on the opposed position of the SNP, and two mutant (MUT) miR-182 and miR-96, in which the G nucleotide was replaced by a C nucleotide. The seed regions (marked in bold) of the mutant miRNAs are fully complimentary to the binding site. FIG. 16 depicts Palladin protein levels 48 hours following transfection of T47D cells by the indicated miRNAs, as determined by western blot analysis. Actin levels were used for normalization.

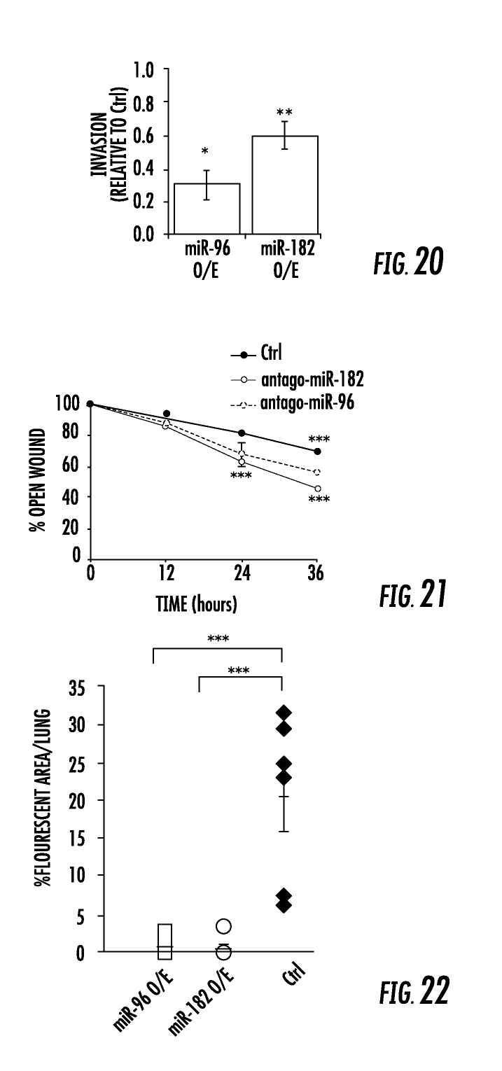

Subsequently, the effect of miR-96 and miR-182 on the invasive behavior of the cells was determined. Migration and invasion abilities were tested using wound-healing assay, transwell migration assay and Matrigel invasion assay. Hs578 cells were transfected by either hsa-miR-182, hsa-miR-96 or pcDNA3 control plasmid (Ctrl). Over-expression of miR-182 inhibited wound closure (by 52.6±21.9% after 20 hours, FIG. 17 (graph of FIG. 17 represents the width of remaining open wound calculated in relation to time 0 (n=3)), and over-expression of miR-96 and miR-182 inhibit invasion in trans-well invasion assay (by 14±4.5% and 25.1±10.5%, respectively, FIG. 18) of Hs578 human invasive breast cancer cells. As shown at FIG. 18, over-expression of hsa-miR-182 and hsa-miR-96 reduced invasion, as demonstrated by a Matrigel invasion assay. Representative fields are on the left of FIG. 18, and the results were calculated as invasion rate in relation to control cells.

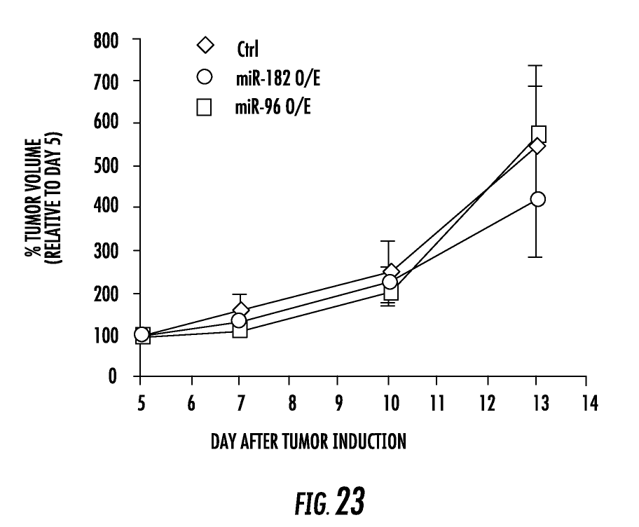

Similarly, stable over-expression of miR-96 and miR-182 inhibited migration in a trans-well migration assay (by 59.5±21.9 and 19.5±8.9%, respectively, as shown at FIG. 19) and invasion in trans-well invasion assay (by 69.8±16.6% and 41±17.9%, respectively, FIG. 20) of mouse 4T1 invasive breast cancer cells. Specifically, transwell migration assay (FIG. 19) and Matrigel invasion assay (FIG. 20) of 4T1 cells stably expressing mmu-miR-182 or mmu-miR-96 showed decreased migration and invasion abilities compared to 4T1 cells stably expressing scrambled sequence as control (Ctrl). Representative fields are on the left. Results were calculated as migration or invasion rates in relation to control cells (n=3).

In contrast, down-regulation of miR-96 and miR-182 enhanced wound closure (by 20.9±4.6% and 33.8±6.6%, respectively, FIG. 21) of MCF-7 non-invasive breast cancer cells. Taken together, these data suggest that miR-96 and miR-182 reduced migration and invasion abilities of breast cancer cells, at least partly through Palladin down-regulation.

### Example 8—In Vivo Experiments

To validate that the effects described at Example 7 also occurred in vivo, an examination was conducted of Palladin and miR-182/96 relation in The Cancer Genome Atlas (TCGA) Breast invasive carcinoma (BRCA) cohort (Cancer Genome Atlas Network. Nature 490, 61-70 (2012)). In agreement with the in vitro findings, a negative correlation was observed between Palladin and miR-96 or miR-182 normalized expression levels (r=−0.3 and r=−0.2, respectively). Unfortunately, there was no indication as to the effect of Palladin on the metastasis state because only a few (n=21) samples had detectable distant organ metastasis (pathologic M1). Yet, an association with lymph nodes metastases was observed when adjusting for tumor size (pathologic T). Both pathologic N staging (N0-N3), and the number of lymph nodes positive by H&E were significantly increased with Palladin expression levels (P≤0.005). These associations, however, could not reliably be explained by miR-96 or miR-182 expression levels, as no significant association was obtained between these miRNAs and lymph nodes metastases (see Table at Example 12).

To explore whether miR-96 and miR-182 could prevent breast cancer metastasis in vivo, the prevalence of metastases was examined in an orthotopic breast cancer mouse model evolved from 4T1 cells that were engineered to overexpress miR-96 or miR-182. 4T1 cells were selected as tumor growth and metastatic spread of these cells in BALB/c mice closely mimic stage IV human breast cancer. A profound decrease in the appearance of lung metastatic nodules was found in tumors stably expressing miR-96 or miR-182 compared to tumors stably expressing the scrambled control (FIG. 22), with no effect on primary tumor characteristics (FIG. 23).

### Example 9—Constructs

For the luciferase reporter assays, fragments of the PALLD 3′-UTR spanning the miRNA-96/182 binding sites were amplified from human genomic DNA, and cloned downstream to the Renilla Luciferase Reporter of the psiCHECK-2 plasmid (Promega) that contain a Firefly Luciferase Reporter (used as control) under a different promoter.

Three Luciferase constructs under regulation of the PALLD 3′-UTR were prepared (FIG. 7): 3′-UTR fragment possessing a G allele in the rs1071738 PALLD SNP position; 3′-UTR fragment possessing a C allele in the rs1071738 PALLD SNP position; negative control 3′-UTR in which the miRNA-96/182-binding site was deleted by restriction enzymes. FIG. 7 is a schematic representation of predicted binding sites for hsa-miR-182/96 on the 3′UTR of PALLD gene. rs1071738 PALLD SNP is marked by the arrow. Three Luciferase constructs under regulation of PALLD 3′UTR were used for transient reporter assay experiments: negative control 3′UTR (Target-deletion), G allele, and C allele. Sequences of mature hsa-miR-182 and hsa-miR-96 aligned to the target site, ‘seed’ region are marked in bold.

For miRNA overexpression, Pre-miRNAs (hsa-miR-96, hsa-miR-182) were cloned into the miRNA expression vector miRVec that was provided by Prof. R. Agami. Vectors expressing mutant hsa-miR-96/182 were generated by mutating the miRVec plasmids expressing WT hsa-miR-96/182 using QuikChange Lightning site-directed mutagenesis kit (Agilent Technologies).

For transient and stable overexpression of mouse miRNA-96/182, Pre-miRNAs (mmu-miR-96/182) were amplified from DNA of 4T1 cells and cloned downstream of the CMV promoter of the CD515B-1_pCDHCMV-MCS-EF1-Hygro Lentivirus Expression Vector (Tarom).

### Example 10—Transfection

HeLa, HEK-293 T, MCF-7, Hs578, T47D, and 4T1 cells were transfected when cells were 50 to 75% confluent. RNA sequences or DNA plasmids were transfected together with a transfection reagent in Opti-MEM serum (Biological Industries). HEK-293 T cells were transfected using TransIT-LT1 Transfection Reagent (Minis) and all other cells were transfected with Lipofectamin 2000 transfection reagent (Invitrogen). For miRNA overexpression studies, 0.5 μg of miRVec plasmid (for human cell lines) or CD515-B plasmid (for murine 4T1 cell line) were transfected. For miRNA inhibition studies, 30 pmole antagomiRs (Ambion) or scrambled control RNA sequence were transfected. GFP was transfected as a control and its detection was confirmed 24 hours following transfection. Cells were harvested for RNA extraction, protein extraction, or lysate preparation 24 to 48 hours following transfection.

### Example 11—Dual Luciferase Reporter Assay

HEK-293 T or HeLa cells were seeded in a 24 wells plate. At about 60% confluence, cells were co-transfected with the 5 ng psiCHECK-2 containing the desired 3′-UTR and 485 ng miRVec containing the desired pre-miRNA. Forty-eight hours following transfection, lysates were extracted and Firefly and Renilla Luciferase activities were measured using the Dual-Luciferase Reporter Assay System kit (Promega) and a Veritas microplate luminometer.

### Example 12—miRNA/mRNA Expression Levels Determination

Total RNA from cell lines was extracted using TRIzol reagent (Invitrogen, Life Technologies). RNA from primary tumor samples was extracted from frozen tissues by homogenization by TissueLyser LT (Qiagen) in TRIzol reagent according to the manufacturer's instructions (Invitrogen, Life Technologies). RNA quality was measured using NanoDrop (Thermo Scientific). cDNA for miRNA and mRNA was synthesized from total RNA.

Reverse transcription reaction for mRNA was conducted with random primer and SuperScript III reverse transcriptase (Invitrogen). Reverse transcription for specific miRNAs was performed with TaqMa miRNA Assays (Applied Biosystems; ABI). Single miRNA/mRNA expression was tested similarly using TaqMan Universal PCR Master Mix (No AmpErase UNG; Applied Biosystems) or SYBR green PCR master mix (Applied Biosystems), respectively, using StepOnePlus real-time PCR system (Applied Biosystems). Specific primer pairs for mRNA expression detection were ordered from Sigma, as shown in the following table:

| Primer name | Sequence |
|---|---|
| hPalladin-For | AACCGAGCAGGACAGAAC |
| hPalladin-Rev | TGGTGGCACTCCCAATAC |
| hGAPDH-For | AGCCACATCGCTGAGACA |
| hGAPDH-Rev | GCCCAATACGACCAAATCC |
| mPalladin-For | AGCATGCACCAGGATAATCA |
| mPalladin-Rev | CAGGACACAATGCCTGCTT |
| m β-Actin-For | ACCAGAGGCATACAGGGACA |
| m β-Actin-Rev | CTAAGGCCAACCGTGAAAAG |

Palladin mRNA quantification was performed by primers that amplify isoforms 1, 3 and 4. Expression values were calculated based on the comparative threshold cycle (Ct) method. miRNA levels were normalized to U6 snRNA and mRNA expression levels were normalized to human GAPDH or mouse Actin.

### Example 13—Western Blot Analysis

Cells were homogenized with lysis buffer, and debris was removed by centrifugation. Protein concentrations were determined using the Bio-Rad protein assay (Bio-Rad Laboratories). Lysates were resolved by SDS-PAGE through 4-12% gels (GeBaGel), and transferred by electroporation to nitrocellulose membrane. Membranes were blocked for 1 hour in TBST buffer containing 5% milk, blotted with anti-Palladin (ProteinTech, cat#10853-1-AP) or anti-Actin (Millipore, clone C4, cat# MAB1501) primary antibodies for 18 hours, followed by a secondary antibody linked to horseradish peroxidase.

The anti-Palladin antibody was generated against the C-terminal 385 amino acids of palladin, and recognized most Palladin isoforms except isoform 6. Immunoreactive bands were detected with enhanced chemiluminescence reagent (Thermo Scientific). Band quantification was performed using ImageJ software (National Institutes of Health) and protein levels were normalized to Actin levels.

### Example 14—Generation of miR-182/96 Stably Expressing Cells

CD515B-1 Lentivectors expressing mmu-miR-96, mmu-miR-182, or a scrambled sequence were prepared as described herein. Packaging was done in HEK-293 T cells with pPACKH1 Lentiviral vector packaging (SBI). Forty-eight hours following HEK-293 transfection, virions containing supernatants were collected. 1 M Hepes (Biological Industries) was added at a 1:20 ratio, supernatants were filtered, supplemented with 5 μL mL⁻¹ polybrene (Sigma) and stored at −80° C. for further use. 4T1-mCherry cells at 50% confluence were infected with the lentiviruses in a six-well plate. Selection was done under the pressure of 200 μg mL⁻¹ Hygromycin (Megapharm).

### Example 15—Generation of Palladin Knock-Down Cells

Palladin knock-down was performed using shRNA sequences (Dharmacon) based on the RNAi Consortium (TRC) by the Broad Institute. The target sequence on Palladin coding region was as follows: 5′-GCTAACCTATGAGGAAAGAAT-3′. Scrambled shRNA sequence was used as a control. The lentiviral vector pLKO.1 was used for shRNA expression. Packaging was done in HEK-293 T cells with ViraPower Lentiviral packaging mix (Invitrogen). Forty-eight hours following HEK-293 transfection, virions containing supernatants were collected and stored at −80° C. Before use, supernatants were filtered and supplemented with 5 μL mL⁻¹ polybrene (Sigma). 4T1 cells at 40% confluence were infected with the lentiviruses in a six-well plate and selection was done under the pressure of 2 μg/mL Puromycin (A.G. Scientific).

### Example 16—Wound Healing Assay

Hs578, MCF-7, or 4T1 (stably expressing mCherry) cells were cultured in complete growth media until about 90% confluence. Cells were conditioned for 5 to 8 hours in DMEM media (Hs578 and MCF-7) or RPMI media (4T1) supplemented with 0.1% FBS, and then adherent cell monolayers were scratched with a 10 μL pipette tip and cultured in complete medium.

Cells were allowed to close the wound for 20 h (Hs578), 24 h (4T1), and 36 h (MCF-7), and were observed under phase-contrast microscopy. 4T1 cells were also observed under fluorescent microscopy (using Nikon Eclipse Ti Epi-fluorescence microscope). The percentage of wound closure was assessed in relation to time 0 by ImageJ software (National Institutes of Health).

### Example 17—Transwell Migration and Invasion Assays

Migration and invasion abilities of breast cancer cells were assessed based on the area covered with cells invading through either transwell inserts (Costar) for migration assays or Matrigel-coated invasion chambers (BD Biosciences), both possessing 8 μm pores. Forty-eight hours following transfection, and 16 hours following starvation in cell culture media supplemented with only 0.1% FBS, Hs578 and 4T1 cells were trypsinized and seeded at 0.5×10⁵ and 1×10⁵ cells per well, respectively, into Transwell chambers (for migration or invasion assays).

4T1 cells stably expressing miRNAs were conditioned overnight in their growth media, supplemented with only 0.1% FBS, and then trypsinized and seeded at 1×10⁵ cells per well into transwell chambers. The lower chamber contained complete media as chemoattractant. Cells were allowed to migrate/invade for 20 to 24 hours, and then wells were fixed with cold Methanol, washed with PBS, and stained by Hemacolor for microscopy (Merck). The non-migrating/invading cells on the upper surface of the insert were removed. The cells that had migrated to the basal side of the membrane were visualized with a Nikon Eclipse Ti microscope at 200× magnification. Pictures of 5 to 10 random fields from three replicate wells were obtained and the percentage of covered area was assessed using ImageJ software.

### Example 18—Cell Proliferation Assay

Proliferation rates for 4T1 and MCF-7 cells were measured using the FITC BrdU Flow Kit (BD Biosciences) according to the manufacturer's instructions. Twenty-four to forty-eight hours following transfection, cells were incubated with Bromodeoxyuridine (BrdU) for 30 minutes. BrdU and DAPI expressions were detected by the Gallios FACS instrument and determined by Flowing software 2. Proliferation rate for Hs578 cells was measured 48 hours following transfection using ViaLight Plus Cell proliferation and cytotoxicity assay (Lonza), according to the manufacturer's instructions.

### Example 19—Statistical Analysis of TCGA BRCA Data

The RNA and miRNA-sequencing and clinical data of BRCA study samples were obtained from The Cancer Genome Atlas (TCGA) Data portal (Level 3, open access)34, and available for 1,203 (mRNA) and 1,176 (miRNA) women, after excluding 12 males. Gene-level transcription estimates in RSEM normalized count were retrieved, and utilized in the statistical analyses. Correlations between normalized transcript counts were measured using the Pearson's method.

ANOVA was used to test the association between Palladin expression and lymph node metastasis while controlling for other staging factors. The reduced model included Palladin expression versus only the pathologic T (T1-4, ordinal), and the full model included pathologic T and pathologic N (NO-3, ordinal), or the number of lymph nodes positive by H&E (discrete). Not included was the pathologic M factor in the models as only 21 subjects had detectable distant organ metastasis (M1), and this exclusion resulted in the use of 999 M0 samples for the analyses herein. Standard residuals of the reduced model were calculated to display the association results. All of the statistical analyses and plots were performed using R programming language.

Access was gained to the TCGA controlled data via ‘The database of Genotypes and Phenotypes’ (dbGaP) to retrieve rs1071738 genotypes (that is, germline). Genotype calls were available for 1,015 subjects (1,011 with normal/tumor pair) from the Affymetrix Genome-Wide Human SNP Array 6.0 (SNP_A-2089440) level 2 data. However, only 460 samples remained after excluding non-Caucasians (about 20%) and samples with missing mRNA and/or miRNA expression levels (about 200 subjects). The power of this sample size was estimated to be insufficient (<30%) by using the QUANTO software package57 (frequency set as 40%, and standardized effect-size as 0.1, typical for SNPs based on genome-wide association studies).

1. A composition comprising:
a metal nanoparticle functionalized with a miRNA and a targeting biomolecule,
wherein the miRNA is configured to bind to a gene at a target site comprising a germline sequence variant, and the targeting biomolecule is configured to bind to a marker that is expressed or overexpressed by a cancer cell.

2. The composition of claim 1, wherein the germline sequence variant comprises a single nucleotide polymorphism.

3. The composition of claim 2, wherein the gene comprises a PALLD gene, and the single nucleotide polymorphism is rs1071738.

4. The composition of claim 1, wherein the gene comprises (i) an ancestral allele that permits miRNA:mRNA binding, and (ii) an alternate allele that disrupts miRNA:mRNA binding; and the miRNA comprises an engineered miRNA configured to bind to the alternate allele.

5. The composition of claim 1, wherein the miRNA comprises a wild-type miR-182, an engineered miR-182, a wild-type miR-96, an engineered miR-96, or a combination thereof.

6. The composition of claim 1, wherein the gene encodes a cytoskeletal protein associated with cell-cell junctions, cell-matrix junctions, or a combination thereof.

7. The composition of claim 6, wherein the cytoskeletal protein is selected from Palladin, Vinculin, or a combination thereof.

8. The composition of claim 1, wherein the gene is selected from PALLD, ROCK2, S100A8, CSF1R, EPHA3, PARVA, PDGFRB, or a combination thereof.

9. The composition of claim 1, wherein the targeting biomolecule comprises a peptide.

10. The composition of claim 9, wherein the peptide is a pentapeptide.

11. The composition of claim 10, wherein the pentapeptide comprises CREKA (Cys-Arg-Glu-Lys-Ala).

12. The composition of claim 1, wherein the marker comprises a fibrin-fibronectin complex.

13. The composition of claim 1, wherein the cancer cell comprises a 4T1 breast cancer cell.

14. The composition of claim 1, wherein the miRNA is present in the composition at an amount of about 200 mols to about 300 mols per metal nanoparticle.

15. The composition of claim 1, wherein the miRNA is present in the composition at an amount of about 250 mols per metal nanoparticle.

16. The composition of claim 1, wherein the targeting peptide is present in the composition at an amount of about 100 mols to about 200 mols per metal nanoparticle.

17. The composition of claim 1, wherein the targeting peptide is present in the composition at an amount of about 145 mols to about 150 mols per metal nanoparticle.

18. The composition of claim 1, wherein the metal nanoparticle is a gold nanoparticle.

19. The composition of claim 1, wherein the metal nanoparticle has an average diameter of about 10 nm to about 100 nm.

20. The composition of claim 1, wherein the metal nanoparticle has an average diameter of about 30 nm to about 50 nm.

21. The composition of claim 1, wherein the metal nanoparticle has an average diameter of about 40 nm.

22. The composition of claim 1, further comprising a drug, wherein the drug is intercalated in the miRNA, conjugated to the metal nanoparticle, or a combination thereof.

23. The composition of claim 1, further comprising a hydrogel in which the metal nanoparticle is dispersed.

24. The composition of claim 23, further comprising a drug, wherein the drug is intercalated in the miRNA, conjugated to the metal nanoparticle, dispersed in the hydrogel, or a combination thereof.

25. The composition of claim 22, wherein the drug comprises one or more chemotherapeutic agents.

26. The composition of claim 25, wherein the drug comprises cisplatin.

27. A method of drug delivery, the method comprising:
providing a first solution comprising a first polymer component comprising a first polymer having one or more aldehydes;
providing a second solution comprising at least one of (i) a dendrimer comprising at least two branches with one or more surface groups, wherein about 25% to 100% of the surface groups comprise at least one primary or secondary amine, and (ii) a second polymer component comprising a second polymer having one or more amines;
combining the first and second solutions together to produce a hydrogel composite; and
contacting one or more biological tissues with the hydrogel composite,
wherein at least one of the first solution and the second solution comprises the composition of claim 1.

28. The method of claim 27, wherein at least one of the first solution and the second solution further comprises a drug.

29. The method of claim 28, wherein the drug comprises one or more chemotherapeutic agents.

30-33. (canceled)

34. The method of claim 27, wherein the first polymer comprises a polysaccharide.

35-36. (canceled)

37. The method of claim 27, wherein the dendrimer comprises a PAMAM dendrimer.

38-39. (canceled)

40. The method of claim 27, wherein the second polymer is a polyamine.

41-44. (canceled)

45. A kit for making a hydrogel composite, the kit comprising:
a first part that includes a first solution comprising a first polymer component comprising a first polymer having one or more aldehydes; and
a second part that includes a second solution comprising at least one of (i) a dendrimer comprising at least two branches with one or more surface groups, wherein about 25% to 100% of the surface groups comprise at least one primary or secondary amine, and (ii) a second polymer component comprising a second polymer having one or more amines;
wherein at least one of the first solution and the second solution comprises the composition of claim 1.

46. The kit of claim 45, wherein at least one of the first solution and the second solution further comprises a drug.

47-48. (canceled)

49. The kit of claim 45, further comprising a syringe, wherein the first solution and the second solution are stored in the syringe.

50. The kit of claim 49, wherein the syringe comprises separate reservoirs for the first solution and the second solution.

51. (canceled)

52. A method for local delivery of a miRNA to a biological tissue, comprising:
applying to the biological tissue the composition of claim 23; and
permitting the metal nanoparticle to diffuse from the composition into the biological tissue.

53. A method of treatment or prophylaxis of cancer in a patient, comprising:
administering to the patient in need thereof an effective amount of the composition of claim 1; and
binding the targeting biomolecule to a cancer cell to permit the miRNA to prevent or reduce the rate of metastasis of the cancer cell.

54. (canceled)

55. A composition comprising:
a metal nanoparticle functionalized with (1) a CREKA pentapeptide, and (2) at least one of a wild-type miR-182, an engineered miR-182, a wild-type miR-96, and an engineered miR-96;
wherein the wild-type miR-182 and the wild-type miR-96 are configured to bind to a binding site of a first allele of a PALLD gene, and the engineered miR-182 and the engineered miR-96 are configured to bind to a binding site of a second allele of the PALLD gene, and wherein the first allele and the second allele differ due to an rs1071738 single nucleotide polymorphism.


SEQUENCE LISTING (18 sequences)

SEQ ID NO: 1 - protein, 5 aa, Artificial Sequence
  Description of Artificial Sequence Synthetic peptide
CREKA

SEQ ID NO: 2 - DNA, 18 bp, Artificial Sequence
  Description of Artificial Sequence Synthetic primer
aaccgagcaggacagaac

SEQ ID NO: 3 - DNA, 18 bp, Artificial Sequence
  Description of Artificial Sequence Synthetic primer
tggtggcactcccaatac

SEQ ID NO: 4 - DNA, 18 bp, Artificial Sequence
  Description of Artificial Sequence Synthetic primer
agccacatcgctgagaca

SEQ ID NO: 5 - DNA, 19 bp, Artificial Sequence
  Description of Artificial Sequence Synthetic primer
gcccaatacgaccaaatcc

SEQ ID NO: 6 - DNA, 20 bp, Artificial Sequence
  Description of Artificial Sequence Synthetic primer
agcatgcaccaggataatca

SEQ ID NO: 7 - DNA, 19 bp, Artificial Sequence
  Description of Artificial Sequence Synthetic primer
caggacacaatgcctgctt

SEQ ID NO: 8 - DNA, 20 bp, Artificial Sequence
  Description of Artificial Sequence Synthetic primer
accagaggcatacagggaca

SEQ ID NO: 9 - DNA, 20 bp, Artificial Sequence
  Description of Artificial Sequence Synthetic primer
ctaaggccaaccgtgaaaag

SEQ ID NO: 10 - DNA, 21 bp, Homo sapiens
gctaacctatgaggaaagaat

SEQ ID NO: 11 - DNA, 17 bp, Homo sapiens
tggaatttttctgaatg

SEQ ID NO: 12 - DNA, 91 bp, Homo sapiens
tggaatttaagataccatacacagtctctcatggacctatctctattgtagaattatgacttatgtcttacttggcaaatttttctgaatg

SEQ ID NO: 13 - DNA, 91 bp, Homo sapiens
tggaatttaagataccatacacagtctctcatggacctatctctattgtagaattatgacttatgtcttacttgccaaatttttctgaatg

SEQ ID NO: 14 - RNA, 24 nt, Homo sapiens
uuuggcaaugguagaacucacacu

SEQ ID NO: 15 - RNA, 23 nt, Homo sapiens
uuuggcacuagcacauuuuugcu

SEQ ID NO: 16 - DNA, 36 bp, Homo sapiens
ttgtagaattatgacttatgtcttacttggcaaatt

SEQ ID NO: 17 - RNA, 24 nt, Homo sapiens
uuugccaaugguagaacucacacu

SEQ ID NO: 18 - RNA, 23 nt, Homo sapiens
uuugccacuagcacauuuuugcu